(19) United States
(12) Patent Application Publication
(10) Pub. No.: US 2007/0104733 A1
(43) Pub. Date: May 10, 2007

(54) TISSUE TARGETED ANTIGENIC ACTIVATION OF THE IMMUNE RESPONSE TO CANCERS

(76) Inventor: HAROLD DAVID GUNN, Vancouver (CA)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/553,972

(22) Filed: Oct. 27, 2006

Related U.S. Application Data
(63) Continuation-in-part of application No. PCT/CA05/00812, filed on May 30, 2005.
(60) Provisional application No. 60/577,206, filed on Jun. 7, 2004.

(51) Int. Cl. A61K 39/12; A61K 39/02

(52) U.S. Cl. 424/204.1; 424/234.1

(57) ABSTRACT

The invention provides in part methods of treating cancers of a specific organ or tissue by administering an composition that is antigenically specific for one or more microbes that are pathogenic in the specific organ or tissue in which the cancer is situated. The formulations of the invention thereby facilitate activation of an immune response to a cancer in a particular tissue or organ. In some embodiments, microbial species of the endogenous flora which are capable of causing infection in the relevant organ or tissue may be used in the formulation of the antigenic compositions. The administration of the immunogenic compositions may be repeated relatively frequently over a relatively long period of time, in a dosage that reproduces a consistent visible inflammatory reaction at the successive site or sites of administration, which may for example be by intradermal or subcutaneous injection.

### FIELD OF THE INVENTION

In various aspects, the invention relates to immunological cancer therapies. In alternative embodiments, the invention provides methods of formulating antigenic microbial composition and methods of using the antigenic compositions to treat cancers.

### BACKGROUND OF THE INVENTION

More than one in three people in the developed nations are diagnosed with cancer. More than one in four die from it. Therapies for cancer have primarily relied upon treatments such as surgery, chemotherapy, and radiation. These approaches however, while beneficial for some types and stages of cancer, have proved to be of limited efficacy in many common types and stages of cancers. For example, surgical treatment of a tumor requires complete removal of cancerous tissue to prevent reoccurrence. Similarly, radiation therapy requires complete destruction of cancerous cells. This is difficult since, in theory, a single malignant cell can proliferate sufficiently to cause reoccurrence of the cancer. Also, both surgical treatment and radiation therapy are directed to localized areas of cancer, and are relatively ineffective when the cancer metastasizes. Often surgery or radiation or both are used in combination with systemic approaches such as chemotherapy. Chemotherapy however has the problem of non-selectivity with the concomitant problem of deleterious side effects, as well as the possibility of the cancer cells developing resistance to the drugs.

Alternative approaches for the treatment of cancers have included therapies that involve stimulation of the immune system such as cytokine therapy (e.g., recombinant interleukin 2 and gamma interferon for kidney cancers), dendritic cell therapy, autologous tumor vaccine therapy, genetically-altered vaccine therapy, lymphocyte therapy, and microbial vaccine therapies. Microbial vaccines have been used to vaccinate subjects against pathogens that are associated with cancer, such as the human papillomavirus. Immunostimulatory microbial vaccines that are not targeted to cancer-causing organisms, i.e. non-specific vaccines, such as pyrogenic vaccines, have a long clinical history that includes reports of successes and failures in treating a variety of cancers. For example, Coley's vaccine (a combination of Streptococcus pyogenes and Serratia marcescens) has been reported to be helpful for the treatment of sarcomas, and lymphomas (Nauts H G, Fowler G M, Bogato F H. A review of the influence of bacterial infection and of bacterial products (Coley's toxins) on malignant tumors in man. Acta Med Scand 1953; 145 (Suppl. 276):5-103). Clinical trials have reportedly demonstrated the benefit of Coley's vaccine treatment for lymphoma and melanoma (Kempin S, Cirrincone C, Myers J et al: Combined modality therapy of advanced nodular lymphomas: the role of nonspecific immunotherapy (MBV) as an important determinant of response and survival. Proc Am Soc Clin Oncol 1983;24:56; Kolmel K F, Vehmeyer K: Treatment of advanced malignant melanoma by a pyrogenic bacterial lysate: a pilot study. Onkologie 1991; 14:411-17).

It has been suggested that the effectiveness of some non-specific bacterial cancer vaccines is attributable to particular bacterial components or products, such as bacterial DNA or endotoxin (LPS), or because they induce the expression of particular factors, such as tumor necrosis factor (TNF) or interleukin-12. A correspondingly broad range of physiological mechanisms have been ascribed to such treatments, ranging from generalized effects of fever to anti-angiogenic mechanisms. In accordance with these various principles, a wide variety of microbial vaccines have been tested as general immune stimulants for the treatment of cancer, many have shown negative results, amongst those that have shown positive results are the following.

Intradermal BCG (Mycobacterium bovis) vaccine treatment has been reported to be effective for the treatment of stomach cancer (Ochiai T, Sato J, Hayashi R, et al: Postoperative adjuvant immunotherapy of gastric cancer with BCG-cell wall endoskeleton. Three- to six-year follow-up of a randomized clinical trial. Cancer Immunol Immunother 1983; 14:167-171) and colon cancer (Smith R E, Colangelo L, Wieand H S, Begovic M, Wolmark N Randomized trial of adjuvant therapy in colon carcinoma: 10-Year results of NSABP protocol C-01. J. NCI 2004; 96(15) 1128-32; Uyl-de Groot C A, Vermorken J B, Hanna M G, Verboon P, Groot M T, Bonsel G J, Meijer C J, Pinedo H M Immunotherapy with autologous tumor cell-BCG vaccine in patients with colon cancer: a prospective study of medical and economic benefits Vaccine 2005; 23(17-18) 2379-87).

Mycobacterium w vaccine therapy, in combination with chemotherapy and radiation, was found to significantly improve quality of life and response to treatment in patients with lung cancer (Sur P, Dastidar A. Role of Mycobacterium w as adjuvant treatment of lung cancer (non-small cell lung cancer). J Indian Med Assoc 2003 February; 101(2):118-120). Similarly, Mycobacterium vaccae vaccine therapy was found to improve quality of life (O'Brien M, Anderson H, Kaukel E, et al. SRL172 (killed Mycobacterium vaccae) in patients with advanced non-small-cell lung cancer: phase III results) and symptom control (Harper-Wynne C, Sumpter K, Ryan C, et al. Addition of SRL 172 to standard chemotherapy in small cell lung cancer (SCLC) improves symptom control. Lung Cancer 2005 February; 47(2):289-90) in lung cancer patients.

Corynebacterium parvum vaccine was linked with a trend towards improved survival for the treatment of melanoma (Balch C M, Smalley R V, Bartolucci A A et al: A randomized prospective trial of adjuvant C. parvum immunotherapy in 260 patients with clinically localized melanoma (stage 1). Cancer 1982 Mar. 15; 49(6):1079-84).

Intradermal Streptococcus pyogenes vaccine therapy was found to be effective for the treatment of stomach cancer (Hanaue H, Kim D Y, Machimura T et al. Hemolytic streptococcus preparation OK-432; beneficial adjuvant therapy in recurrent gastric carcinoma. Tokai J Exp Clin Med 1987 November; 12(4):209-14).

Nocardia rubra vaccine was found to be effective for the treatment of lung cancer (Yasumoto K, Yamamura Y. Randomized clinical trial of non-specific immunotherapy with cell-wall skeleton of Nocardia rubra. Biomed Pharmacother 1984; 38(1):48-54; Ogura T. Immunotherapy of respectable lung cancer using Nocardia rubra cell wall skeleton. Gan To Kagaku Ryoho 1983 February; 10(2 Pt 2):366-72) and linked to a trend to improved survival for the treatment acute myelogenous leukemia (Ohno R, Nakamura H, Kodera Y, et al. Randomized controlled study of chemoimmunotherapy of acute myelogenous leukemia (AML) in adults with Nocardia rubra cell-wall skeleton and irradiated allogeneic AML cells. Cancer 1986 Apr. 15; 57(8):1483-8).

Lactobacillus casei vaccine treatment combined with radiation was found to more effective for the treatment of cervical cancer than radiation alone.

Pseudomonas aeruginosa vaccine treatment was found to increase the effectiveness of chemotherapy in the treatment of lymphoma and lung cancer (Li Z, Hao D, Zhang H, Ren L, et al. A clinical study on PA_MSHA vaccine used for adjuvant therapy of lymphoma and lung cancer. Hua Xi Yi Ke Da Xue Xue Bao 2000 September; 31 (3):334-7).

Childhood vaccination with the smallpox vaccine (i.e., Vaccinia) was found to be associated with a decreased risk of melanoma later in life (Pfahlberg A, Kolmel K F, Grange J M. et al. Inverse association between melanoma and previous vaccinations against tuberculosis and smallpox: results of the FEBIM study. J Invest Dermatol 2002(119) 570-575) as well as decreased mortality in those patients who did develop melanoma (Kolmel K F, Grange J M, Krone B, et al. Prior immunization of patients with malignant melanoma with vaccinia or BCG is associated with better survival. An European Organization for Research and Treatment of Cancer cohort study on 542 patients. Eur J Cancer 41(2005) 118-125).

Rabies virus vaccine was found to result in temporary remission in 8 of 30 patients with melanoma (Higgins G, Pack G: Virus therapy in the treatment of tumors. Bull Hosp Joint Dis 1951; 12:379-382; Pack G: Note on the experimental use of rabies vaccine for melanomatosis. Arch Dermatol 1950; 62:694-695).

In spite of substantial efforts to engage the immune system to combat cancers, there is little epidemiological evidence of widespread success in improving the survival of cancer patient populations. While it has been recognized that immunostimulatory approaches have promise, it has also been recognized that significant challenges characterize the field (“Endotoxin and Exotoxin Induced Tumor Regression with Special Reference to Coley Toxins: A Survey of the Literature and Possible Immunological Mechanisms “Report to the National Cancer Institute Office of Alternative and Complementary Medicine August 1997. Ralf Kleef, Mary Ann Richardson, Nancy Russell, Cristina Ramirez). There remains a need for alternative immunological treatments for cancers.

### SUMMARY OF THE INVENTION

In one aspect, the invention provides methods for formulating an immunogenic composition for treating a cancer situated in a specific organ or tissue in a mammal, such as human patient. The method may include selecting at least one microbial pathogen that is pathogenic in the organ or tissue of the mammal within which the cancer is situated. An antigenic composition may be produced that includes antigenic determinants that together are specific for or characteristic of the microbial pathogen.

The antigenic composition may be formulated for subcutaneous or intradermal injection, to produce the immunogenic composition. A diagnostic step may be used to identify the specific organ or tissue within which the cancer is situated, prior to producing the antigenic composition. The antigenic composition may be formulated for producing localized skin inflammation at a site of administration, for example an area of inflammation from 2 mm to 80 mm in diameter. The site of the cancer may be a primary site, or a secondary site of metastasis. The antigenic composition may be formulated for repeated subcutaneous or intradermal administration, for example at alternating successive sites. The microbial pathogen may be a bacterial species, such as a species that is endogenous to flora of the patient or an exogenous species that causes infection in the organ or tissue. The antigenic composition may be sufficiently specific that it is capable of eliciting an adaptive immune response in the mammal specific to the microbial pathogen, such as a killed bacterial composition. In alternative embodiments, the microbial pathogen may be a virus. Immunogenic compositions of the invention may also be formulated or administered with anti-inflammatory modalities, such as an NSAID.

In alternative embodiments, the invention involves methods of treating a mammal for a cancer situated in a tissue or an organ. The treatment may for example anticipate the development of the cancer in the tissue, for example if the site of a primary tumour suggests the likelihood of metastasis to a particular tissue or organ, then the patient may be prophylactically treated for that metastasis. The method may include administering to the subject an effective amount of an antigenic composition comprising antigenic determinants that together are specific for at least one microbial pathogen. The microbial pathogen may be pathogenic in the specific organ or tissue of the mammal within which the cancer is situated. The antigenic composition may be administered at an administration site in successive doses given at a dosage interval, for example of between one hour and one month, over a dosage duration, for example of at least two weeks, two months, six months, one year or two years. Each dose may be metered so that it is effective to cause visible localized inflammation at the administration site.

The invention provides in part methods of treating cancers of a specific organ or tissue in a subject by administering one or more antigens of one or more microbial pathogens, such as bacterial or viral species that are pathogenic in the specific organ or tissue.

In alternative embodiments, the pathogenic microbial species may be capable of causing infection naturally, (i.e. without human intervention) when present in the specific organ or tissue in a healthy subject, or may have caused an infection in the specific organ or tissue in a healthy subject. In alternative embodiments, the antigen may be administered by administering a whole microbial species. In alternative embodiments, the method may for example include administering at least two or more microbial species, or administering at least three or more microbial species, and the microbes may be bacteria or viruses. In alternative embodiments, the method may further include administering a supplement or an adjuvant. In alternative embodiments, the administering may elicit an immune response in said subject.

A “cancer” or “neoplasm,” as used herein, is any unwanted growth of cells serving no physiological function. In general, a cancer cell has been released from its normal cell division control, i.e., a cell whose growth is not regulated by the ordinary biochemical and physical influences in the cellular environment. Thus, “cancer” is a general term for diseases characterized by abnormal uncontrolled cell growth. In most cases, a cancer cell proliferates to form clonal cells that are either benign or malignant. The resulting lump or cell mass, “neoplasm” or “tumor,” is generally capable of invading and destroying surrounding normal tissues. By “malignancy” is meant an abnormal growth of any cell type or tissue, that has a deleterious effect in the organism having the abnormal growth. The term “malignancy” or “cancer” includes cell growths that are technically benign but which carry the risk of becoming malignant. Cancer cells may spread from their original site to other parts of the body through the lymphatic system or blood stream in a process known as “metastasis.” Many cancers are refractory to treatment and prove fatal. Examples of cancers or neoplasms include, without limitation, transformed and immortalized cells, tumors, carcinomas, in various organs and tissues as described herein or known to those of skill in the art.

A “cell” is the basic structural and functional unit of a living organism. In higher organisms, e.g., animals, cells having similar structure and function generally aggregate into “tissues” that perform particular functions. Thus, a tissue includes a collection of similar cells and surrounding intercellular substances, e.g., epithelial tissue, connective tissue, muscle, nerve. An “organ” is a fully differentiated structural and functional unit in a higher organism that may be composed of different types tissues and is specialized for some particular function, e.g., kidney, heart, brain, liver, etc. Accordingly, by “specific organ, tissue, or cell” is meant herein to include any particular organ, and to include the cells and tissues found in that organ.

An “infection” is the state or condition in which the body or a part of it is invaded by a pathogenic agent (e.g., a microbe, such as a bacterium) which, under favorable conditions, multiplies and produces effects that are injurious (Taber's Cyclopedic Medical Dictionary, 14th Ed., C. L. Thomas, Ed., F. A. Davis Company, PA, USA). An infection may not always be apparent clinically, or may result in localized cellular injury. Infections may remain localized, subclinical, and temporary if the body's defensive mechanisms are effective. A local infection may persist and spread to become an acute, a subacute, or a chronic clinical infection or disease state. A local infection may also become systemic when the pathogenic agent gains access to the lymphatic or vascular system (On-Line Medical Dictionary, http://cancerweb.ncl.ac.uk/omd/). Localized infection is usually accompanied by inflammation, but inflammation may occur without infection.

“Inflammation” is the characteristic tissue reaction to injury (marked by swelling, redness, heat, and pain), and includes the successive changes that occur in living tissue when it is injured. Infection and inflammation are different conditions, although one may arise from the other (Taber's Cyclopedic Medical Dictionary, supra). Accordingly, inflammation may occur without infection and infection may occur without inflammation (although inflammation typically results from infection by pathogenic bacteria).

Inflammation is characterized by the following symptoms: redness (rubor), heat (calor), swelling (tumor), pain (dolor). Localized visible inflammation on the skin may be apparent from a combination of these symptoms, particularly redness at a site of administration.

Various subjects may be treated in accordance with alternative aspects of the invention. A “subject” is an animal, e.g, a mammal, to whom the specific pathogenic bacteria, bacterial antigens, or compositions thereof of the invention may be administered. Accordingly, a subject may be a patient, e.g., a human, suffering from a cancer, or suspected of having a cancer, or at risk for developing a cancer. A subject may also be an experimental animal, e.g., an animal model of a cancer. In some embodiments, the terms “subject” and “patient” may be used interchangeably, and may include a human, a non-human mammal, a non-human primate, a rat, mouse, dog, etc. A healthy subject may be a human who is not suffering from a cancer or suspected of having a cancer, or who is not suffering from a chronic disorder or condition. A “healthy subject” may also be a subject who is not immunocompromised. By immunocompromised is meant any condition in which the immune system functions in an abnormal or incomplete manner, for example, a condition which prevents or reduces a full and normal immune response, or renders the immunocompromised subject more susceptible to microbial (e.g., bacterial) infection. Immunocompromisation may be due to disease, certain medications, or conditions present at birth. Immunocompromised subjects may be found more frequently among infants, children, the elderly, individuals undergoing extensive drug or radiation therapy.

An “immune response” includes, but is not limited to, one or more of the following responses in a mammal: induction of antibodies, neutrophils, monocytes, macrophages, B cells, T cells (including helper T cells, natural killer cells, cytotoxic T cells, γδ T cells) directed specifically to the antigen(s) in a composition or vaccine, following administration of the composition or vaccine. An immune response to a composition or vaccine thus generally includes the development in the host animal of a cellular and/or antibody-mediated response to the composition or vaccine of interest. In general, the immune response will result in slowing or stopping the progression of cancer in the animal.

### BRIEF DESCRIPTION OF THE DRAWINGS

FIG. 1 shows a survival curve for patients diagnosed with stage 3B or 4 inoperable lung cancer (all patients), comparing patients treated with MRV, patients not treated with the MRV, and a standard SEER survival curve.

FIG. 2 shows a survival curve for patients diagnosed with stage 3B or 4 inoperable lung cancer (patients treated for at least 2 months with MRV), comparing patients treated with MRV, patients not treated with the MRV, and a standard SEER survival curve.

FIG. 3 shows a survival curve for patients diagnosed with lung cancer, illustrating the benefits of treatment with the MRV composition of the invention, comparing patients treated with MRV, patients not treated with the MRV, and a standard SEER survival curve.

FIG. 4 shows a survival curve for patients diagnosed with lung cancer, illustrating the effect of treatments for at least 2 months, comparing patients treated with MRV, patients not treated with the MRV, and a standard SEER survival curve.

FIG. 5 shows a survival curve for patients diagnosed with lung cancer, illustrating the effect of treatments for at least 6 months duration, comparing patients treated with MRV, patients not treated with the MRV, and a standard SEER survival curve.

FIG. 6 shows a survival curve for a cumulative series of 52 breast cancer patients with metastases to bone and/or lung, comparing patients treated with MRV, patients not treated with the MRV, and a standard SEER survival curve.

FIG. 7 is a comparison of survival of a cumulative series of metastatic prostate cancer patients who had surgery or radiation to destroy their prostate gland (and thus, the primary tumour) and who had detectable cancer limited to bone metastases, comparing patients treated with MRV, patients not treated with the MRV, and a standard SEER survival curve.

FIG. 8 shows a survival curve for patients initially diagnosed with Stage 4 colorectal cancer, comparing patients treated with PVF, patients treated with MRV, patients not treated with an antigenic composition and a standard SEER survival curve.

FIG. 9 shows a survival curve for patients initially diagnosed with Stage 4 Colorectal Cancer, with date from patients receiving treatment within 3 months of diagnosis, comparing patients treated with PVF, patients treated with MRV, patients not treated with an antigenic composition and a standard SEER survival curve.

### DETAILED DESCRIPTION OF THE INVENTION

In various aspects, the invention relates to the surprising discovery that administration of microbial pathogens that are pathogenic in a particular tissue or organ is effective in treating cancer situated of that specific tissue or organ. Accordingly, the invention provides antigenic compositions, including whole, killed bacterial species, as well as components thereof, for the treatment of cancer and methods for using the same.

Based on observations from treating patients, it was found that administering compositions including many of the most common pathogenic bacteria that cause lung and upper respiratory tract infection and/or including Staphylococcus aureus, one of the most common causes of skin and breast infection, and septicemia, was surprisingly and unexpectedly effective in improving the clinical course of cancer of the lung, bone, breast, skin, perineum, multiple myeloma and lymphoma (cancer of the lymph glands) and malignant melanoma (a type of skin cancer). Similarly, it was surprisingly and unexpectedly found that administering a composition including pathogenic Escherchia coli, which is a common cause of colon, kidney, bladder, prostate, uterine, peritoneal and ovarian infection was effective in improving the clinical course of cancer in the colon, liver, abdominal lymph nodes, ovary and peritoneum.

These results indicate that a composition including antigens of pathogenic microbial species that most commonly cause infection in a particular tissue or organ will be the most effective formulation for treating a cancer in that tissue or organ. For example, cancer in the lung is most effectively treated with a microbial composition including pathogenic species that commonly cause lower respiratory tract infection, while cancer in the kidney is most effectively treated with a composition including pathogenic microbial species that commonly cause kidney infections.

Antigenic compositions of the invention may be produced that include antigenic determinants that together are specific for or characteristic of a microbial pathogen. In this context, by “specific”, it is meant that the antigenic determinants are sufficiently characteristic of the pathogen that they could be used to raise an adaptive immune response against the pathogen in the patient, if the antigenic determinants were to be administered in an appropriate manner to have that effect. The antigenic determinants need not be so specific that they are characteristic of only the particular strain or species of pathogen, since an adaptive immune response against the pathogen may be cross reactive to other closely related organisms.

In some embodiments, the compositions of pathogenic microbes may be used for treating primary cancer sites and/or sites of metastasis. Thus, for example, the microbial compositions may be used for the treatment of a cancer at a particular site, regardless of whether the cancer is the primary cancer or the metastatic site. The composition may be directed to the treatment of each cancer site, or may be a combined composition for both the primary cancer and the metastatic site(s). For example, to treat kidney cancer that has metastasized to the lung and bone, three different compositions including pathogenic species that are pathogenic in kidney, lung and bone, or a combined composition thereof may be used. In embodiments, the compositions may be administered in different locations at the same time or at different times.

For example, for lung cancer with metastasis to the bone, in alternative embodiments, both a microbial composition including bacteria which commonly cause lung infection and a microbial composition including bacteria which commonly cause bone infection may be used. Similarly, for colon cancer with metastasis to the lungs, both a pathogenic bacterial composition including bacteria which commonly cause colon infection and a microbial composition including bacteria which commonly cause lung infection may be used; for prostate cancer with metastasis to the bones, both a pathogenic bacterial composition including bacteria which commonly cause prostate infection and a pathogenic bacterial composition including bacteria that commonly cause bone infection may be used.

The following list provides some non-limiting examples of primary cancers and their common sites for secondary spread (metastases):

| Primary cancer | Common sites for metastases |
|---|---|
| prostate | bone, lungs |
| breast | bone, lungs, skin, liver, brain |
| lung | bone, brain, liver, lungs |
| colon | liver, lungs, bone, brain |
| kidney | lungs, bone, brain |
| pancreas | liver, lungs |
| melanoma | lungs, skin, liver, brain |
| uterus | lungs, bones, ovaries |
| ovary | liver, lung |
| bladder | bone, lung, liver |
| head and neck | bone, lungs |
| sarcoma | lungs, brain |
| stomach | liver |
| cervix | bone, lungs |
| testes | lungs |
| thyroid | bone, lungs |

In some embodiments, the antigenic compositions may be used for treating or preventing cancers at primary sites or preventing metastasis. For example, in long-term smokers, an antigenic composition specific for cancer of the lung (for example including antigenic determinants of bacteria which commonly cause lung infection) may be used to appropriately stimulate the immune system to defend against the development of cancer within the lung tissue. As another example, an antigenic composition specific for cancer of the breast (for example including antigenic determinants of bacteria which commonly cause breast infection) could be used to prevent breast cancer in women with a strong family history of breast cancer or a genetic predisposition. In alternative embodiments, an antigenic composition including bacteria which commonly cause bone infection may be used to treat bone metastases in a patient with prostate cancer. In further alternative embodiments, an antigenic composition including bacteria which commonly cause lung infection may be used to treat lung metastases in a patient with malignant melanoma.

Various alternative embodiments and examples of the invention are described herein. These embodiments and examples are illustrative and should not be construed as limiting the scope of the invention.

Cancers

Most cancers fall within three broad histological classifications: carcinomas, which are the predominant cancers and are cancers of epithelial cells or cells covering the external or internal surfaces of organs, glands, or other body structures (e.g., skin, uterus, lung, breast, prostate, stomach, bowel), and which tend to metastasize; sarcomas, which are derived from connective or supportive tissue (e.g., bone, cartilage, tendons, ligaments, fat, muscle); and hematologic tumors, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumors (mixed connective tissue types). Hematologic tumors may be myelomas, which originate in the plasma cells of bone marrow; leukemias which may be “liquid cancers” and are cancers of the bone marrow and may be myelogenous or granulocytic leukemia (myeloid and granulocytic white blood cells), lymphatic, lymphocytic, or lymphoblastic leukemias (lymphoid and lymphocytic blood cells) or polycythemia vera or erythremia (various blood cell products, but with red cells predominating); or lymphomas, which may be solid tumors and which develop in the glands or nodes of the lymphatic system, and which may be Hodgkin or Non-Hodgkin lymphomas. In addition, mixed type cancers, such as adenosquamous carcinomas, mixed mesodermal tumors, carcinosarcomas, or teratocarcinomas also exist.

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body, that is different from the primary site. With the present invention, treatment is directed to the site of the cancer, not type of cancer, so that a cancer of any type that is situated in the lung, for example, would be treated on the basis of this localization in the lung.

Cancers named based on primary site may be correlated with histological classifications. For example, lung cancers are generally small cell lung cancers or non-small cell lung cancers, which may be squamous cell carcinoma, adenocarcinoma, or large cell carcinoma; skin cancers are generally basal cell cancers, squamous cell cancers, or melanomas. Lymphomas may arise in the lymph nodes associated with the head, neck and chest, as well as in the abdominal lymph nodes or in the axillary or inguinal lymph nodes. Identification and classification of types and stages of cancers may be performed by using for example information provided by the Surveillance, Epidemiology, and End Results (SEER) Program of the National Cancer Institute, which is an authoritative source of information on cancer incidence and survival in the United States and is recognized around the world. The SEER Program currently collects and publishes cancer incidence and survival data from 14 population-based cancer registries and three supplemental registries covering approximately 26 percent of the US population. The program routinely collects data on patient demographics, primary tumor site, morphology, stage at diagnosis, first course of treatment, and follow-up for vital status, and is the only comprehensive source of population-based information in the United States that includes stage of cancer at the time of diagnosis and survival rates within each stage. Information on more than 3 million in situ and invasive cancer cases is included in the SEER database, and approximately 170,000 new cases are added each year within the SEER coverage areas. The incidence and survival data of the SEER Program may be used to access standard survival for a particular cancer site and stage. For example, to ensure an optimal comparison group, specific criteria may be selected from the database, including date of diagnosis and exact stage (in the case of the lung cancer example herein, the years were selected to match the time-frame of the retrospective review, and stage 3B and 4 lung cancer were selected).

Bacteria and Bacterial Colonizations and Infections

Most animals are colonized to some degree by extrinsic organisms, such as bacteria, which generally exist in symbiotic or commensal relationships with the host animal. Thus, many species of generally harmless bacteria are normally found in healthy animals, and are usually localized to specific organs and tissues. Often, these bacteria aid in the normal functioning of the body. For example, in humans, symbiotic Lactobacillus acidophilus may be found in the intestine, where they assist in food digestion.

Bacteria that are generally harmless, such as Lactobacillus acidophilus, can cause infection in healthy subjects, with results ranging from mild to severe infection to death. Whether or not a bacterium is pathogenic (i.e., causes infection) depends to some extent on factors such as the route of entry and access to specific host cells, tissues, or organs; the intrinsic virulence of the bacterium; the amount of the bacteria present at the site of potential infection; or the immune status of the host animal (e.g., healthy or immunocompromised). Thus, bacteria that are normally harmless can become pathogenic given favorable conditions for infection, and even the most virulent bacterium requires specific circumstances to cause infection. Accordingly, microbial species that are members of the normal flora can be pathogens, when they move beyond their normal ecological role in the endogenous flora. For example, endogenous species can cause infection outside of their ecological niche in regions of anotomical proximity, for example by contiguous spread.

Pathogenic bacteria and viruses generally cause infections in specific cells, tissues, or organs (e.g., localized infections) in otherwise healthy subjects. Examples of pathogenic bacteria and viruses that commonly cause infections in specific organs and tissues of the body are listed below; it will be understood that these examples are not intended to be limiting and that a skilled person would be able to readily recognize and identify infectious or pathogenic bacteria that cause infections in various organs and tissues in healthy adults (and recognize the relative frequency of infection with each bacterial species) based on the knowledge in the field as represented, for example, by the following publications: Manual of Clinical Microbiology 8th Edition, Patrick Murray, Ed., 2003, ASM Press American Society for Microbiology, Washington D.C., USA; Mandell, Douglas, and Bennett's Principles and Practice of Infectious Diseases 5th Edition, G. L. Mandell, J. E. Bennett, R. Dolin, Eds., 2000, Churchill Livingstone, Philadelphia, Pa., USA, all of which are incorporated by reference herein.

Infections of the skin are commonly caused by the following bacterial species: Staphylococcus aureus, Beta hemolytic streptococci group A, B, C or G, Corynebacterium diptheriae, Corynebacterium ulcerans, or Pseudomonas aeruginosa; or viral pathogens: rubeola, rubella, varicella-zoster, echoviruses, coxsackieviruses, adenovirus, vaccinia, herpes simplex, or parvo Bg9.

Infections of the soft tissue (e.g., fat and muscle) are commonly caused by the following bacterial species: Streptococcus pyogenes, Staphylococcus aureus, Clostridium perfringens, or other Clostridium spp. or viral pathogens: influenza, or coxsackieviruses

Infections of the breast are commonly caused by the following bacterial species: Staphylococcus aureus, or Streptococcus pyogenes.

Infections of the lymph nodes of the head and neck are commonly caused by the following bacterial species: Staphylococcus aureus, or Streptococcus pyogenes; or viral pathogens: Epstein-Barr, cytomegalovirus, adenovirus, measles, rubella, herpes simplex, coxsackieviruses, or varicella-zoster.

Infections of the lymph nodes of the arm/axillae are commonly caused by the following bacterial species: Staphylococcus aureus, or Streptococcus pyogenes; or viral pathogens: measles, rubella, Epstein-Barr, cytomegalovirus, adenovirus, or varicella-zoster.

Infections of the lymph nodes of the mediastinum are commonly caused by the following bacterial species: viridans streptococci, Peptococcus spp., Peptostreptococcus spp., Bacteroides spp., or Fusobacterium; or viral pathogens: measles, rubella, Epstein-Barr, cytomegalovirus, varicella-zoster, or adenovirus.

Infections of the intra-abdominal lymph nodes are commonly caused by the following bacterial species: Yersinia enterocolitica, Yersinia pseudotuberculosis, Salmonella spp., Streptococcus pyogenes, Escherichia coli, or Staphylococcus aureus; or viral pathogens: measles, rubella, Epstein-Barr, cytomegalovirus, varicella-zoster, adenovirus, influenza, or coxsackieviruses.

Infections of the lymph nodes of the leg/inguinal region are commonly caused by the following bacterial species: Staphylococcus aureus, or Streptococcus pyogenes; or viral pathogens: measles, rubella, Epstein-Barr, cytomegalovirus, or herpes simplex.

Infections of the blood (i.e., septicemia) are commonly caused by the following bacterial species: Staphylococcus aureus, Streptococcus pyogenes, coagulase-negative staphylococci, Enterococcus spp., Escherichia coli, Klebsiella spp., Enterobacter spp., Proteus spp., Pseudomonas aeruginosa, Bacteroides fragilis, Streptococcus pneumoniae, or group B streptococci; or viral pathogens: rubeola, rubella, varicella-zoster, echoviruses, coxsackieviruses, adenovirus, Epstein-Barr, or cytomegalovirus.

Infections of the bone are commonly caused by the following bacterial species: Staphylococcus aureus, coagulase-negative staphylococci, Streptococcus pyogenes, Streptococcus pneumoniae, Streptococcus agalactiae, other streptococci spp., Escherichia coli, Pseudomonas spp., Enterobacter spp., Proteus spp., or Serratia spp.; or viral pathogens: parvovirus B19, rubella, or hepatitis B.

Infections of the meninges are commonly caused by the following bacterial species: Haemophilus influenzae, Neisseria meningitidis, Streptococcus pneumoniae, Streptococcus agalactiae, or Listeria monocytogenes; or viral pathogens: echoviruses, coxsackieviruses, other enteroviruses, or mumps.

Infections of the brain are commonly caused by the following bacterial species: Streptococcus spp. (including S. anginosus, S. constellatus, S. intermedius), Staphylococcus aureus, Bacteroides spp., Prevotella spp., Proteus spp., Escherichia coli, Klebsiella spp., Pseudomonas spp., Enterobacter spp., or Borrelia burgdorferi; or viral pathogens: coxsackieviruses, echoviruses, poliovirus, other enteroviruses, mumps, herpes simplex, varicella-zoster, flaviviruses, or bunyaviruses.

Infections of the spinal cord are commonly caused by the following bacterial species: Haemophilus influenzae, Neisseria meningitidis, Streptococcus pneumoniae, Streptococcus agalactiae, Listeria monocytogenes, or Borrelia burgdorferi; or viral pathogens: coxsackieviruses, echoviruses, poliovirus, other enteroviruses, mumps, herpes simplex, varicella-zoster, flaviviruses, or bunyaviruses.

Infections of the eye/orbit are commonly caused by the following bacterial species: Staphylococcus aureus, Streptococcus pyogenes, Streptococcus pneumoniae, Streptococcus milleri, Escherichia coli, Bacillus cereus, Chlamydia trachomatis, Haemophilus influenza, Pseudomonas spp., Klebsiella spp., or Treponema pallidum; or viral pathogens: adenoviruses, herpes simplex, varicella-zoster, or cytomegalovirus.

Infections of the salivary glands are commonly caused by the following bacterial species: Staphylococcus aureus, viridans streptococci (e.g., Streptococcus salivarius, Streptococcus sanguis, Streptococcus mutans), Peptostreptococcus spp., or Bacteroides spp., or other oral anaerobes; or viral pathogens: mumps, influenza, enteroviruses, or rabies.

Infections of the mouth are commonly caused by the following bacterial species: Prevotella melaminogenicus, anaerobic streptococci, viridans streptococci, Actinomyces spp., Peptostreptococcus spp., or Bacteroides spp., or other oral anaerobes; or viral pathogens: herpes simplex, coxsackieviruses, or Epstein-Barr.

Infections of the tonsils are commonly caused by the following bacterial species: Streptococcus pyogenes, or Group C or G B-hemolytic streptococci; or viral pathogens: rhinoviruses, influenza, coronavirus, adenovirus, parainfluenza, respiratory syncytial virus, or herpes simplex.

Infections of the sinuses are commonly caused by the following bacterial species: Streptococcus pneumoniae, Haemophilus influenza, Moraxella catarrhalis, α-streptococci, anaerobic bacteria (e.g., Prevotella), or Staphylococcus aureus; or viral pathogens: rhinoviruses, influenza, adenovirus, or parainfluenza.

Infections of the nasopharynx are commonly caused by the following bacterial species: Streptococcus pyogenes, or Group C or G B-hemolytic streptococci; or viral pathogens: rhinoviruses, influenza, coronavirus, adenovirus, parainfluenza, respiratory syncytial virus, or herpes simplex.

Infections of the thyroid are commonly caused by the following bacterial species. Staphylococcus aureus, Streptococcus pyogenes, or Streptococcus pneumoniae; or viral pathogens: mumps, or influenza.

Infections of the larynx are commonly caused by the following bacterial species: Mycoplasma pneumoniae, Chlamydophila pneumoniae, for Streptococcus pyogenes; or viral pathogens: rhinovirus, influenza, parainfluenza, adenovirus, corona virus, or human metapneumovirus.

Infections of the trachea are commonly caused by the following bacterial species: Mycoplasma pneumoniae; or viral pathogens: parainfluenza, influenza, respiratory syncytial virus, or adenovirus.

Infections of the bronchi are commonly caused by the following bacterial species: Mycoplasma pneumoniae, Chlamydophila pneumoniae, Bordetella pertussis, Streptococcus pneumoniae, or Haemophilus influenzae; or viral pathogens: influenza, adenovirus, rhinovirus, coronavirus, parainfluenza, respiratory syncytial virus, human metapneumovirus, or coxsackievirus.

Infections of the lung are commonly caused by the following bacterial species: Streptococcus pneumoniae, Moraxella catarrhalis, Mycoplasma pneumoniae, Klebsiella pneumoniae, Haemophilus influenza, or Staphylococcus aureus; or viral pathogens: influenza, adenovirus, respiratory syncytial virus, or parainfluenza.

Infections of the pleura are commonly caused by the following bacterial species: Staphylococcus aureus, Streptococcus pyogenes, Streptococcus pneumoniae, Haemophilus influenzae, Bacteroides fragilis, Prevotella spp., Fusobacterium nucleatum, peptostreptococcus, or Mycobacterium tuberculosis; or viral pathogens: influenza, adenovirus, respiratory syncytial virus, or parainfluenza.

Infections of the mediastinum are commonly caused by the following bacterial species: viridans streptococci, Peptococcus spp., Peptostreptococcus spp., Bacteroides spp., or Fusobacterium spp.; or viral pathogens: measles, rubella, Epstein-Barr, or cytomegalovirus.

Infections of the heart are commonly caused by the following bacterial species: Streptococcus spp. (including S. mitior, S. bovis, S. sanguis, S. mutans, S. anginosus), Enterococcus spp., Staphylococcus spp., Corynebacterium diptheriae, Clostridium perfringens, Neisseria meningitidis, or Salmonella spp.; or viral pathogens: enteroviruses, coxsackieviruses, echoviruses, poliovirus, adenovirus, mumps, rubeola, or influenza.

Infections of the esophagus are commonly caused by the following bacterial species: Actinomyces spp., Mycobacterium avium, Mycobacterium tuberculosis, or Streptococcus spp.; or viral pathogens: cytomegalovirus, herpes simplex, or varicella-zoster.

Infections of the stomach are commonly caused by the following bacterial species: Streptococcus pyogenes; or viral pathogens; cytomegalovirus, herpes simplex, Epstein-Barr, rotaviruses, noroviruses, or adenoviruses.

Infections of the small bowel are commonly caused by the following bacterial species: Escherichia coli, Clostridium difficile, Bacteroides fragilis, Bacteroides vulgatus, Bacteroides thetaiotaomicron, Clostridium perfringens, Salmonella enteriditis, Yersinia enterocolitica, or Shigella flexneri; or viral pathogens: adenoviruses, astroviruses, caliciviruses, noroviruses, rotaviruses, or cytomegalovirus.

Infections of the colon/rectum are commonly caused by the following bacterial species: Escherichia coli, Clostridium difficile, Bacteroides fragilis, Bacteroides vulgatus, Bacteroides thetaiotaomicron, Clostridium perfringens, Salmonella enteriditis, Yersinia enterocolitica, or Shigella flexneri; or viral pathogens: adenoviruses, astroviruses, caliciviruses, noroviruses, rotaviruses, or cytomegalovirus.

Infections of the anus are commonly caused by the following bacterial species. Streptococcus pyogenes, Bacteroides spp., Fusobacterium spp., anaerobic streptococci, Clostridium spp., E. coli, Enterobacter spp., Pseudomonas aeruginosa, or Treponema pallidum; or viral pathogens: herpes simplex.

Infections of the perineum are commonly caused by the following bacterial species: Escherichia coli, Klebsiella spp., Enterococcus spp., Bacteroides spp., Fusobacterium spp., Clostridium spp., Pseudomonas aeruginosa, anaerobic streptococci, Clostridium spp., E. coli, or Enterobacter spp.; or viral pathogens: herpes simplex.

Infections of the liver are commonly caused by the following bacterial species: Escherichia coli, Klebsiella spp., Streptococcus (anginosus group), Enterococcus, spp. other viridans streptococci, or Bacteroides spp.; or viral pathogens: hepatitis A, Epstein-Barr, herpes simplex, mumps, rubella, rubeola, varicella-zoster, coxsackieviruses, or adenovirus.

Infections of the gallbladder are commonly caused by the following bacterial species: Escherichia coli, Klebsiella spp., Enterobacter spp., enterococci, Bacteroides spp., Fusobacterium spp., Clostridium spp., Salmonella enteriditis, Yersinia enterocolitica, or Shigella flexneri.

Infections of the biliary tract are commonly caused by the following bacterial species: Escherichia coli, Klebsiella spp., Enterobacter spp., enterococci, Bacteroides spp., Fusobacterium spp., Clostridium spp., Salmonella enteriditis, Yersinia enterocolitica, or Shigella flexneri; or viral pathogens: hepatitis A, Epstein-Barr, herpes simplex, mumps, rubella, rubeola, varicella-zoster, cocsackieviruses, or adenovirus.

Infections of the pancreas are commonly caused by the following bacterial species: Escherichia coli, Klebsiella spp., Enterococcus spp., Pseudomonas spp., Staphylococcal spp., Mycoplasma, Salmonella typhi, Leptospirosis spp., or Legionella spp.; or viral pathogens: mumps, coxsackievirus, hepatitis B, cytomegalovirus, herpes simplex 2, or varicella-zoster.

Infections of the spleen are commonly caused by the following bacterial species: Streptococcus spp., Staphylococcus spp., Salmonella spp., Pseudomonas spp., Escherichia coli, or Enterococcus spp.; or viral pathogens: Epstein-Barr, cytomegalovirus, adenovirus, measles, rubella, coxsackieviruses, or varicella-zoster.

Infections of the adrenal gland are commonly caused by the following bacterial species: Streptococcus spp., Staphylococcus spp., Salmonella spp., Pseudomonas spp., Escherichia coli, or Enterococcus spp.; or viral pathogens: varicella-zoster.

Infections of the kidney are commonly caused by the following bacterial species: Escherichia coli, Proteus mirabilis, Proteus vulgatus, Providentia spp., Morganella spp., Enterococcus faecalis, or Pseudomonas aeruginosa; or viral pathogens: BK virus, or mumps.

Infections of the ureter are commonly caused by the following bacterial species: Escherichia coli, Proteus mirabilis, Proteus vulgatus, Providentia spp., Morganella spp., or Enterococcus spp.

Infections of the bladder are commonly caused by the following bacterial species: Escherichia coli, Proteus mirabilis, Proteus vulgatus, Providentia spp., Morganella spp., Enterococcus faecalis, or Corynebacterium jekeum; or viral pathogens: adenovirus, or cytomegalovirus.

Infections of the peritoneum are commonly caused by the following bacterial species: Staphylococcus aureus, Streptococcus pyogenes, Streptococcus pneumonia, Escherichia coli, Klebsiella spp., Proteus spp., enterococci, Bacteroides fragilis, Prevotella melaminogenica, Peptococcus spp., Peptostreptococcus spp., Fusobacterium, or Clostridium spp.

Infections of the retroperitoneal area are commonly caused by the following bacterial species: Escherichia coli, or Staphylococcus aureus.

Infections of the prostate are commonly caused by the following bacterial species: Escherichia coli, Klebsiella spp., Enterobacter spp., Proteus mirabilis, enterococci, Pseudomonas spp., Corynebacterium spp., or Neisseria gonorrhoeae; or viral pathogens: herpes simplex.

Infections of the testicle are commonly caused by the following bacterial species: Escherichia coli, Klebsiella pneumoniae, Pseudomonas aeruginosa, Staphylococcus spp., Streptococcus spp., or Salmonella enteriditis; or viral pathogens: mumps, coxsackievirus, or lymphocytic choriomeningitis virus.

Infections of the penis are commonly caused by the following bacterial species: Staphylococcus aureus, Streptococcus pyogenes, Neisseria gonorrhoeae, or Treponema pallidum; or viral pathogens: herpes simplex.

Infections of the ovary/adnexae are commonly caused by the following bacterial species: Neisseria gonorrhoeae, Chlamydia trachomatis, Gardenerella vaginalis, Prevotella spp., Bacteroides spp., Peptococcus spp. Streptococcus spp., or Escherichia coli.

Infections of the uterus are commonly caused by the following bacterial species: Neisseria gonorrhoeae, Chlamydia trachomatis, Gardenerella vaginalis, Prevotella spp., Bacteroides spp., Peptococcus spp., Streptococcus spp., or Escherichia coli.

Infections of the cervix are commonly caused by the following bacterial species: Neisseria gonorrhoeae, Chlamydia trachomatis, or Treponema pallidum; or viral pathogens: herpes simplex.

Infections of the vagina are commonly caused by the following bacterial species: Gardenerella vaginalis, Prevotella spp., Bacteroides spp., peptococci spp., Escherichia coli, Neisseria gonorrhoeae, Chlamydia Trachomatis, or Treponema pallidum; or viral pathogens: herpes simplex.

Infections of the vulva are commonly caused by the following bacterial species: Staphylococcus aureus, Streptococcus pyogenes, or Treponema pallidum; or viral pathogens: herpes simplex.

Bacterial Strains/Viral Subtypes

It will be understood by a skilled person that bacterial species are classified operationally as collections of similar strains (which generally refers to groups of presumed common ancestry with identifiable physiological but usually not morphological distinctions, and which may be identified using serological techniques against bacterial surface antigens). Thus, each bacterial species (e.g., Streptococcus pneumoniae) has numerous strains (or serotypes), which differ in their ability to cause infection or differ in their ability to cause infection in a particular organ/site. For example, although there are at least 90 serotypes of Streptococcus pneumoniae, serotypes 1, 3, 4, 7, 8, and 12 are most frequently responsible for pneumococcal disease in humans.

As a second example, certain strains of Escherichia coli, referred to as extraintestinal pathogenic E. coli (ExPEC), are more likely to cause urinary tract infection or other extraintestinal infections such as neonatal meningitis, whereas other strains, including enterotoxigenic E. coli (ETEC), enteropathogenic E. coli (EPEC), enterohemorrhagic E. coli (EHEC), Shiga toxin-producing E. coli (STEC), enteroaggregative E. coli (EAEC), enteroinvasive E. coli (EIEC) and diffuse adhering E. coli (DAEC) are more likely to cause gastrointestinal infection/diarrhea. Even among the sub-category of ExPEC strains, specific virulence factors (e.g., production of type-1 fimbriae) enable certain strains to be more capable of causing infection of the bladder, while other virulence factors (e.g., production of P fimbriae) enable other strains to be more capable of causing infection in the kidneys. In accordance with the present invention, an ExPEC strain(s) that is more likely to cause infection in the bladder would be chosen for a formulation to target bladder cancer, whereas an ExPEC strain(s) that is more likely to cause infection in the kidney would be chosen for a formulation to target kidney cancer. Likewise, an ETEC, EPEC, EHEC, STEC, EAEC, EIEC or DAEC strain of E. coli (i.e, strains that cause colon infection), would be chosen for a formulation to treat colon cancer.

Similarly, there may be numerous subtypes of specific viruses. For example, there are three types of influenza viruses, influenza A, influenza B and influenza C, which differ in epidemiology, host range and clinical characteristics. For example, influenza A is more likely to be associated with viral lung infection, whereas influenza B is more likely to be associated with myositis (i.e., muscle infection). Furthermore, each of these three types of influenza virus have numerous subtypes, which also may differ in epidemiology, host range and clinical characteristics. In accordance with the present invention, one would choose an influenza A subtype most commonly associated with lung infection to target lung cancer, whereas one would choose an influenza B strain most commonly associated with myositis to treat cancer of the muscle/soft tissues.

It is understood that a clinical microbiologist skilled in the art would therefore be able to select, based on the present disclosure and the body of are relating to bacterial strains for each species of bacteria (and viral subtypes for each type of virus), the strains of a particular bacterial species (or subtype of a particular virus) to target a specific organ or tissue.

Bacterial Compositions, Dosages, And Administration

The compositions of the invention include antigens of pathogenic microbial (bacterial or viral) species that are pathogenic in a specific tissue or organ. The compositions may include whole bacterial species, or may include extracts or preparations of the pathogenic bacterial species of the invention, such as cell wall or cell membrane extracts or whole cell extracts. The compositions may also include one or more isolated antigens from one or more of the pathogenic bacterial species of the invention; in some embodiments, such compositions may be useful in situations where it may be necessary to precisely administer a specific dose of a particular antigen, or may be useful if administering a whole bacterial species or components thereof (e.g., toxins) may be harmful. Pathogenic bacterial species may be available commercially (from, for example, ATCC (Manassas, Va., USA), or may be clinical isolates from subjects having a bacterial infection of a tissue or organ (e.g., pneumonia).

The microbial compositions of the invention can be provided alone or in combination with other compounds (for example, nucleic acid molecules, small molecules, peptides, or peptide analogues), in the presence of a liposome, an adjuvant, or any pharmaceutically acceptable carrier, in a form suitable for administration to mammals, for example, humans, As used herein “pharmaceutically acceptable carrier” or “excipient” includes any and all solvents, dispersion media, coatings, antibacterial and antifungal agents, isotonic and absorption delaying agents, and the like that are physiologically compatible. The carrier can be suitable for any appropriate form of administration, including subcutaneous, intradermal, intravenous, parenteral, intraperitoneal, intramuscular, sublingual, inhalational, intratumoral or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound (i.e., the specific bacteria, bacterial antigens, or compositions thereof of the invention), use thereof in the pharmaceutical compositions of the invention is contemplated. Supplementary active compounds can also be incorporated into the compositions.

If desired, treatment with bacterial antigens according to the invention may be combined with more traditional and existing therapies for cancer, such as chemotherapy, radiation therapy, surgery, etc., or with any other therapy intended to stimulate the immune system or otherwise benefit the subject, such as nutrients, vitamins and supplements. For example, antioxidants, vitamins A, D, E, C, and B complex; selenium; zinc; co-enzyme Q10, beta carotene, fish oil, curcumin, green tea, bromelain, resveratrol, ground flaxseed; garlic; lycopene; milk thistle; melatonin; cimetidine; indomethacin; or COX-2 Inhibitors (e.g., Celebrex (celecoxib) or Vioxx (rofecoxib)) may be also be administered to the subject.

Conventional pharmaceutical practice may be employed to provide suitable formulations or compositions to administer the compounds to subjects suffering from a cancer. Any appropriate route of administration may be employed, for example, parenteral, intravenous, intradermal, subcutaneous, intramuscular, intracranial, intraorbital, ophthalmic, intraventricular, intracapsular, intraspinal, intrathecal, intracisternal, intraperitoneal, intranasal, inhalational, aerosol, topical, intratumoral or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences” (20th edition), ed. A. Gennaro, 2000, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. For therapeutic or prophylactic compositions, the pathogenic bacterial species are administered to an individual in an amount effective to stop or slow progression or metastasis of the cancer, or to increase survival of the subject (relative to for example prognoses derived from the SEER database) depending on the disorder.

An “effective amount” of a pathogenic microbial species or antigen thereof according to the invention includes a therapeutically effective amount or a prophylactically effective amount. A “therapeutically effective amount” refers to an amount effective, at dosages and for periods of time necessary, to achieve the desired therapeutic result, such as reduction or elimination of the cancer cells or tumors, prevention of carcinogenic processes, slowing the growth of the tumour, or an increase in survival time beyond that which is expected using for example the SEER database. A therapeutically effective amount of a pathogenic microbial (bacterial or viral) species or antigen(s) thereof may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the pathogenic bacterial species or antigen thereof are outweighed by the therapeutically beneficial effects. A “prophylactically effective amount” refers to an amount effective, at dosages and for periods of time necessary, to achieve the desired prophylactic result, such as reduction or elimination of the cancer cells, tissues, organs, or tumors, or an increase in survival time beyond that which is expected using for example the SEER database. Typically, a prophylactic dose is used in subjects prior to or at an earlier stage of cancer, so that a prophylactically effective amount may be less than a therapeutically effective amount. An exemplary range for therapeutically or prophylactically effective amounts of one or more pathogenic bacterial species may be about 1 million to 20,000 million organisms per ml, or may be 100 million to 7000 million organisms per ml, or may be 500 million to 6000 million organisms per ml, or may be 1000 million to 5000 million organisms per ml, or may be 2000 million to 4000 million organisms per ml, or any integer within these ranges. The total concentration of bacteria per ml may range from 20 million to 8000 million organisms per ml, or may be 50 million to 7000 million organisms per ml, or may be 100 million to 6000 million organisms per ml, or may be 500 million to 5000 million organisms per ml, or may be 1000 million to 4000 million organisms per ml, or any integer within these ranges. The range for therapeutically or prophylactically effective amounts of antigens of a pathogenic bacterial species may be any integer from 0.1 nM-0.1M, 0.1 nM-0.05M, 0.05 nM-15 μM or 0.01 nM-10 μM.

It is to be noted that dosage concentrations and ranges may vary with the severity of the condition to be alleviated, or may vary with the subject's immune response. In general, the goal is to achieve an adequate immune response (e.g, a local skin reaction, e.g, from 0.25 inch to 4 inch; or a systemic fever response immune response (e.g., systemic symptoms of fever and sweats). The dose required to achieve an appropriate immune response may vary depending on the individual (and their immune system) and the response desired. Standardized dosages may also be used. For example, if the goal is to achieve a 2 inch local skin reaction, the total bacterial composition dose may range from 2 million bacteria (i.e., 0.001 ml of a vaccine with a concentration of 2,000 million organisms per ml) to more than 4000 million bacteria (i.e., 2 ml of a vaccine with a concentration of 2,000 million organisms per ml). The concentrations of individual bacterial species or antigens thereof within a composition may also be considered, since individuals may vary in their response to various bacterial species. For example, if the concentration of one particular pathogenic bacterial species, cell size of that species or antigen thereof is much higher relative to the concentrations of other pathogenic bacterial species in the vaccine, then the local skin reaction of an individual may be likely due to its response to this specific bacterial species. In some embodiments, the immune system of an individual may respond more strongly to one bacterial species within a composition than another, so the dosage or composition may be adjusted accordingly for that individual.

For any particular subject, specific dosage regimens may be adjusted over time (e.g, daily, every other day, weekly, monthly) according to the individual need and the professional judgement of the person administering or supervising the administration of the compositions. For example, the compositions may be administered every second day. An initial dose of approximately 0.05 ml may be administered subcutaneously, followed by increases from 0.01-0.02 ml every second day until an adequate skin reaction was achieved at the injection site (a 1 inch to 2 inch reaction of visible redness). Once this reaction was achieved, this dosing was continued as a maintenance dose. The maintenance dose may be adjusted from time to time to achieve the desired visible skin reaction (inflammation) at the injection site.

Dosage ranges set forth herein are exemplary only and do not limit the dosage ranges that may be selected by medical practitioners. The amount of active compound (e.g., pathogenic bacterial species or antigens thereof) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In the case of antigenic formulations (analogous to a vaccine), an immunogenically effective amount of a compound of the invention can be provided, alone or in combination with other compounds, with an immunological adjuvant. The compound may also be linked with a carrier molecule, such as bovine serum albumin or keyhole limpet hemocyanin to enhance immunogenicity. An antigenic composition (“vaccine”) is a composition that includes materials that elicit a desired immune response. An antigenic composition may select, activate or expand memory B, T cells, neutrophils, monocytes or macrophages of the immune system to, for example, reduce or eliminate the growth or proliferation of cancerous cells or tissue. In some embodiments, the specific pathogenic microbe, virus, bacteria, bacterial antigens, or compositions thereof of the invention are capable of eliciting the desired immune response in the absence of any other agent, and may therefore be considered to be an antigenic composition. In some embodiments, an antigenic composition includes a suitable carrier, such as an adjuvant, which is an agent that acts in a non-specific manner to increase the immune response to a specific antigen, or to a group of antigens, enabling the reduction of the quantity of antigen in any given vaccine dose, or the reduction of the frequency of dosage required to generate the desired immune response. A bacterial antigenic composition may include weakened (attenuated) or dead bacteria capable of inducing an immune response against the disease or infection normally caused by the bacteria. In some embodiments, an antigenic composition may include live bacteria that are of less virulent strains, and therefore cause a less severe infection.

An antigenic composition comprising killed bacteria may be made as follows. The bacteria may be grown in suitable media, and washed with physiological salt solution. The bacteria may then be centrifuged, resuspended in salt solution, and killed with phenol. The suspensions may be standardized by direct microscopic count, mixed in required amounts, and stored in appropriate containers, which may be tested for safety, shelf life, and sterility in an approved manner. In addition to the pathogenic bacterial species and/or antigens thereof, a killed bacterial vaccine suitable for administration to humans may include 0.4% phenol preservative, 0.9% sodium chloride. Optionally, the bacterial vaccine may also include trace amounts of brain heart infusion (beef), peptones, yeast extract, agar, sheep blood, dextrose, and/or sodium phosphate. The bacterial vaccine may be used for subcutaneous injection.

In antigenic compositions comprising bacteria (analogous to killed bacterial vaccines), the concentrations of specific bacterial species may be about 1 million to 20,000 million organisms per ml, or may be 100 million to 7000 million organisms per ml, or may be 500 million to 6000 million organisms per ml, or may be 1000 million to 5000 million organisms per ml, or may be 2000 million to 4000 million organisms per ml, or any integer within these ranges. The total concentration of bacteria per ml may range from 20 million to 8000 million organisms per ml, or may be 50 million to 7000 million organisms per ml, or may be 100 million to 6000 million organisms per ml, or may be 500 million to 5000 million organisms per ml, or may be 1000 million to 4000 million organisms per ml, or any integer within these ranges.

In some embodiments, an optimal killed bacterial vaccine for lung cancer may be:

| | bacteria per ml | |
|---|---|---|
| Streptococcus pneumoniae | 600 | million |
| Moraxella catarrhalis | 400 | million |
| Mycoplasma pneumoniae | 400 | million |
| Klebsiella pneumoniae | 200 | million |
| Haemophilus influenzae | 200 | million |
| Staphylococcus aureus | 200 | million |
| total: | 2,000 | million |

In some embodiments, an antigenic microbial composition for treating cancer at a particular site (e.g., cancer of the lung) may include specific strain or serotypes that most commonly cause infection (e.g., pneumonia) in that tissue or organ.

In general, the pathogenic bacterial species and antigens thereof of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

In some aspects, the invention involves the use of an anti-inflammatory in conjunction with vaccinations. In these embodiments, a wide variety of anti-inflammatory treatments may be employed, including effective amounts of non-steroidal anti-inflammatory drugs (NSAIDs), including but not limited to: diclofenac potassium, diclofenac sodium, etodolac, indomethicin, ketorolac tromethamine, sulindac, tometin sodium, celecoxib, meloxicam, valdecoxib, floctafenine, mefenamic acid, nabumetone, meloxicam, piroxicam, tenoxicam, fenoprofen calcium, flubiprofen, ibuprofen, ketoprofen, naproxen, naproxen sodium, oxaprozin, tiaprofenic acid, acetylsalicylic acid, diflunisal, choline magnesium trisalicylate, choline salicylate, triethanolamine salicylate, COX1 inhibitors, COX2 inhibitors (e.g. Vioxx, and Celebrex). A variety of herbs and natural health products may also be used to provide anti-inflammatory treatment, including but not limited to: green tea, fish oil, resveratrol, turmeric, bromelain, boswellia, feverfew, quercetin, ginger, rosemary, oregano, cayenne, clove, nutmeg, willowbark. Alternative anti-inflammatory modalities may also include lifestyle modifications, such as: exercise, weight loss, smoking cessation, stress reduction, seeking social support, treatment of depression, stress management, abdominal breath work and dietary change (such as adopting a Mediterranean diet, a low glycemic diet, eating non-charred foods, including foods having omega-3 fatty acids).

### EXAMPLE 1

### Clinical Studies

Bacterial Compositions

Three mixed, killed bacterial compositions have been used to treat a wide variety of cancer types and stages in blinded studies, as follows:

1. The Bayer Corporation MRV™“Bayer MRV” (Hollister-Steir Laboratories, Spokane, Wash., U.S.A.), containing the following bacterial species:

| | Organisms per ml | |
|---|---|---|
| Staphylococcus aureus | 1200 | million |
| viridans and non-hemolytic Streptococci | 200 | million |
| Streptococcus pneumoniae | 150 | million |
| Moraxella (Neisseria) catarrhalis | 150 | million |
| Klebsiella pneumoniae | 150 | million |
| Haemophilus influenzae | 150 | million |

This vaccine was produced for the following indications: rhinitis, infectious asthma, chronic sinusitis, nasal polyposis and chronic serous otitis media. Cancer treatment was not indicated as an intended use for this vaccine. The vaccine also included the following ingredients: 0.4% phenol, 0.9% NaCl, trace amounts of brain heart infusion (beef), peptones, yeast extract, agar, sheep blood, dextrose, and sodium phosphates.]

2. Stallergenes MRV “Stallergenes MRV” (Laboratories des Stallergenes, S.A., Fresnes, France, containing the following:

| | Organisms per ml |
|---|---|
| Staphylococcus aureus | 600 million |
| Staphylococcus albus | 600 million |
| non-hemolytic Streptococci | 200 million |
| Streptococcus pneumoniae | 150 million |
| Moraxella (Neisseria) catarrhalis | 150 million |
| Klebsiella pneumoniae | 150 million |
| Haemophilus influenzae | 150 million |

This vaccine was produced for the same indications as the MRV vaccine i.e., recurrent respiratory tract infections, and listed cancer as a contraindication.

3, Polyvaccinum Forte (PVF; Biomed S.A., Krakow, Poland), containing the following:

| | Organisms per ml |
|---|---|
| Staphylococcus aureus | 500 million |
| Staphylococcus epidermidis | 500 million |
| Escherichia coli | 200 million |
| Corynebacterium pseudodiphtheriticum | 200 million |
| Streptococcus pyogenes | 100 million |
| Streptococcus salivarius (viridans Streptococci) | 100 million |
| Streptococcus pneumoniae | 100 million |
| Moraxella (Neisseria) catarrhalis | 100 million |
| Klebsiella pneumoniae | 100 million |
| Haemophilus influenzae | 100 million |

This vaccine was produced for chronic and recurrent inflammatory conditions of the upper and lower respiratory tract, including rhinopharyngitis, recurrent laryngitis, tracheitis, bronchitis, otitis media: chronic and recurrent neuralgia of trigeminal and occipital nerve, ischialgia, brachial plexitis and intercostais neuralgia, as well as chronic cystoureteritis, vaginitis, adnexitis, and endometrium inflammation. Cancer treatment was not indicated as an intended use for this vaccine.

Of note, although the total concentration of bacteria in PVF is identical to that of the MRVs (Bayer and Stallergenes), patients typically demonstrated a visible inflammatory response to subcutaneous injection of the PVF composition at a much smaller dose than the usual dose required to achieve a similar skin response with the MRV composition, indicating that the reaction was likely occurring to one of the novel components in the Polyvaccinum Forte vaccine, such as E. coli. As set out below, PVF has been found to be effective in the treatment of colon and pancreatic cancers.

Administration

The bacterial compositions (vaccines) were a suspension of killed bacterial cells and therefore, the suspensions were gently shaken prior to use to ensure uniform distribution prior to withdrawing dose from vial, and administered subcutaneously three times a week on Mondays, Wednesdays, and Fridays for at least 6 months. The dose of vaccine required was determined by the adequacy of the immune reaction to the vaccine. Beginning with a very small dose (0.05 cc), the dose was gradually increased (by 0.01-0.02 cc each time) until an adequate immune reaction was achieved. The goal was to achieve a one to two inch diameter round patch of pinkness/redness at the injection site, indicating adequate immune stimulation. Once this reaction was achieved, the dose was maintained at the level required to achieve this reaction. If the reaction was significantly less than two inches (e.g., half an inch) the dose was increased, if it was significantly more than two inches (e.g., three inches), the dose was decreased. This local reaction generally occurs within the first 24 hours after the injection. Patients were asked to check for this reaction and, if present, to measure or mark it. The maintenance dose required to achieve an adequate immune reaction varies considerably, depending on the individual's immune response—as little as 0.001 cc for some people, as much as 2 cc for others. The maximum dose given is 0.40 cc. The vaccine must be stored in a refrigerator (2° to 8° C.). The usual site for injection is the upper arms, the thighs or the abdomen. The exact site of each injection was varied so that it was not given in sites in which pinkness/redness was still present. There are no known contraindications to the vaccines.

Cancer of the Lung

This section relates to primary cancer in the lung, or metasteses to the lung, treated with microbial pathogens of the lung, such as bacteria that are endogenous or exogenous to the lung, or viruses.

Patients qualified for the lung cancer study if they were initially diagnosed with stage 3B or 4-lung (inoperable) cancer. Lung cancer staging was performed using standard methods as for example described in AJCC: Cancer Staging Handbook (sixth edition) 2002; Springer-Verlag New York: Editors: Fredrick Greene, David Page and Irvin Fleming, or in International Union Against Cancer: TNM Classification of Malignant Tumors (sixth edition) 2002; Wiley-Liss Geneva Switzerland: Editors: L. H. Sobin and C. H. Wittekind. For example, lung cancers may be classified as follows:

TNM Lung Clinical and Pathological Classification

T Primary Tumour

TX Primary tumour cannot be assessed, or tumour proven by the presence of malignant cells in sputum or bronchial washings but not visualized by imaging or bronchoscopy
Tis Carcinoma in situ
T0 No evidence of primary tumour
T1 Tumour 3 cm or less in greatest dimension, surrounded by lung or visceral pleura, without bronchoscopic evidence of invasion more proximal than the lobar bronchus (ie, not in the main bronchus)
T2 Tumour with any of the following features of size or extent: More than 3 cm in greatest dimension Involves main bronchus, 2 cm or more distal to the carina Invades visceral pleura Associated with atelectasis or obstructive pneumonitis that extends to the hilar region but does not involve the entire lung
T3 Tumour of any size that directly invades any of the following: chest wall (including superior sulcus tumours), diaphragm, mediastinal pleura, parietal pericardium; or tumour in the main bronchus less than 2 cm distal to the carina but without involvement of the carina; or associated atelectasis or obstructive pneumonitis of the entire lung
T4 Tumour of any size that invades any of the following: mediastinum, heart, great vessels, trachea, esophagus, vertebral body, carina; or tumour with a malignant pleural or pericardial effusion; or with separate tumour nodule(s) within the ipsilateral primary-tumour lobe of the lung,
   N Regional Lymph Nodes
NX Regional lymph nodes cannot be assessed
N0 No regional lymph node metastasis
N1 Metastasis in ipsilateral peribronchial and/or ipsilateral hilar lymph nodes and intrapulmonary nodes, including involvement by direct extension
N2 Metastasis in ipsilateral mediastinal and/or subcarinal lymph node(s)
N3 Metastasis in contralateral mediastinal, contralateral hilar, ipsilateral or contralateral scalene, or supraclavicular lymph node(s)
   M Distant Metastasis
MX Distant metastasis cannot be assessed
M0 No distant metastasis
M1 Distant metastasis; includes separate tumour nodule(s) in the non-primary-tumour lobe (ipsilateral or contralateral)

Stage Grouping of TNM Subsets:

| | | | |
|---|---|---|---|
| Occult | TX | N0 | M0 |
| carcinoma | | | |
| Stage 0 | Tis | N0 | M0 |
| Stage IA | T1 | N0 | M0 |
| Stage IB | T2 | N0 | M0 |
| Stage IIA | T1 | N1 | M0 |
| Stage IIB | T2 | N1 | M0 |
| | T3 | N0 | M0 |
| Stage IIIA | T3 | N1 | M0 |
| | T1 | N2 | M0 |
| | T2 | N2 | M0 |
| | T3 | N2 | M0 |
| Stage IIIB | Any T | N3 | M0 |
| | T4 | Any N | M0 |
| Stage IV | Any T | Any N | M1 |

Charts with diagnostic codes 162.9 (lung cancer) and 197 (metastatic cancer) were collected manually and electronically. Information was collected on these patients, such as date of diagnosis, date of death, and cancer stage. Charts for patients were reviewed to confirm the date of diagnosis and cancer stage. Patients were excluded from the analysis for the following reasons: 1) wrong stage; 2) missing data; 3) no chart, or; 4) chart did not reach in time for the data analysis. 20 patients were excluded from the study because their charts have not arrived yet or there was insufficient information, of which 6 were MRV users. The study group includes 108 patients in total: 50 who took the MRV vaccine and 58 who did not take the MRV vaccine.

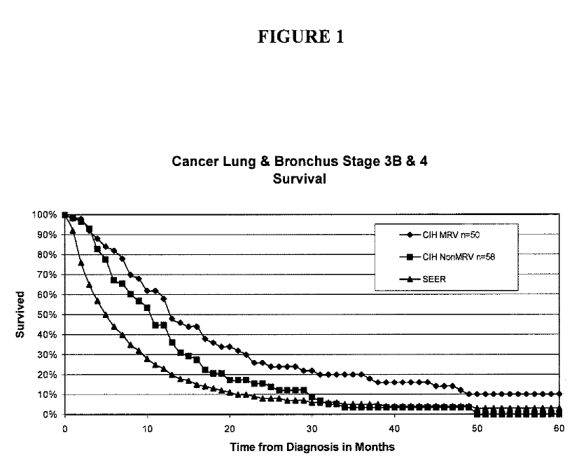

Comparison of survival of patients initially diagnosed with stage 3B and 4 lung cancer who took MRV with patients who didn't take MRV and with SEER standard survival data for patients initially diagnosed with stage 3B and 4 lung cancer (FIG. 1) was as follows:

| | SEER | non-MRV | MRV |
|---|---|---|---|
| median survival: | 5 months | 10.5 months | 12.5 months |
| survival at 1 year: | 25% | 45% | 58% |
| survival at 3 years: | 5% | 3% | 20% |
| survival at 5 years: | 3% | 0% | 10% |

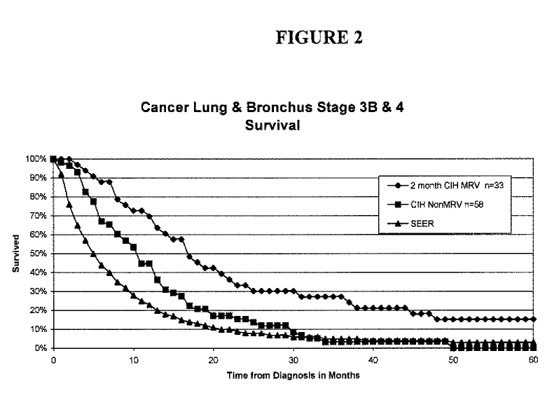

A comparison of survival (as above), including only those patients who took MRV for at least 2 months (FIG. 2) is as follows:
median survival: 16.5 months
survival at 1 year: 70%
survival at 3 years: 27%
survival at 5 years. 15%

Median survival and survival at 1 year, 3 years and 5 years, was substantially better in the group that was treated with MRV (containing bacteria which commonly cause lung infection), evidence of the effectiveness of this vaccine for the treatment of lung cancer. Patients who were treated with the MRV vaccine for more than 2 months had higher survival rates, further evidence of the effectiveness of this vaccine for the treatment of lung cancer.

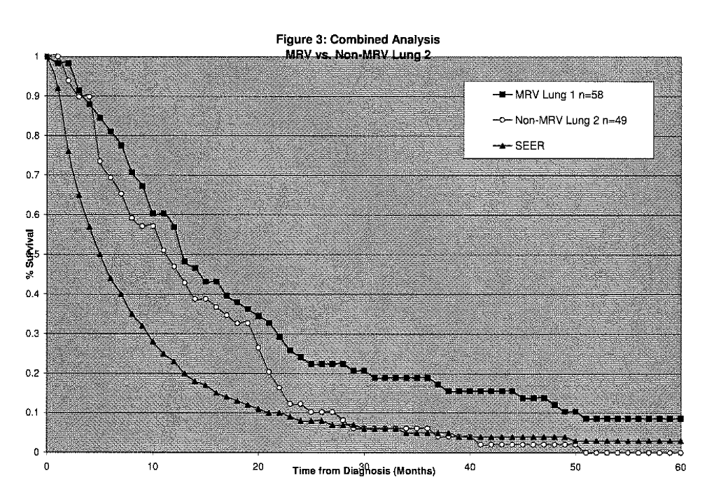

An alternative analysis was conducted on data that included a patient population to whom the MRV composition was not available, to address a perceived potential for bias caused by sicker patients being more likely to choose the novel treatment (with MRV) and healthier patients being potentially less likely to submit to the use of the antigenic compositions of the invention. Comparison of survival of MRV patients to whom the MRV composition was available (designated “Lung 1”) to survival of non-MRV patients to whom the MRV composition was not available (designated “Lung 2”) removes some of this selection bias, providing a clearer and more accurate illustration of the benefit of MRV treatment, as illustrated in FIG. 3.

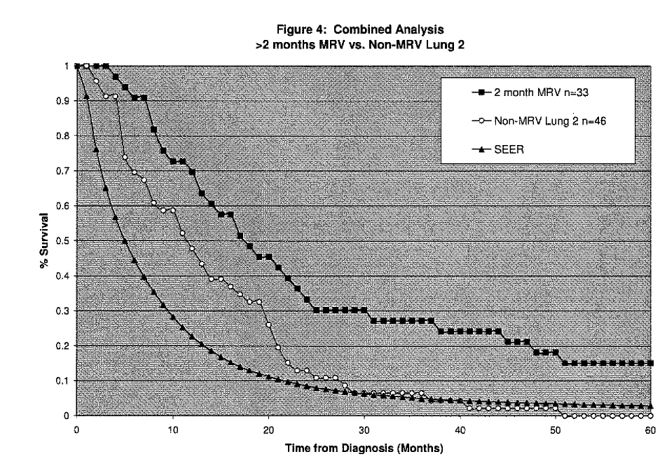

In some embodiments, particularly striking clinical benefits have been obtained with antigenic bacterial compositions used in repeated frequent injections (i.e., three times per week) for a prolonged period of time—such as at least 2, 3, 4, 5, 6 or 12 months (in the context of advanced cancer such as inoperable lung cancer, the longer periods may be most beneficial). Treatments of this kind may be carried out so as to provide sustained, prolonged immune stimulation. When the above analysis is restricted to patients who were treated with MRV for a minimum of 2 months, the survival advantage of MRV treatment is even more clearly illustrated FIG. 4.

As illustrated in FIG. 4, one-year survival of inoperable lung cancer patients treated with MRV for at least two months was 70%, compared to just 48% for the non-MRV Lung 2 group and 23% for the SEER database group. 3-year survival of the MRV group was more than 4 times that of both the non-MRV patients and the SEER registry. None of the non-MRV group in the Lung 2 study survived for 5 years, whereas 15% of patients treated with MRV for a minimum two-month period were still alive 5 years after diagnosis. In the context of an illness such as inoperable lung cancer that is considered terminal and has a usual 5-year survival rate of only 1.2% (SEER registry), the above results are extremely encouraging and surprising. Accordingly, in some embodiments, cancers, such as advanced cancers, such as inoperable lung cancer, may be treated over a dosing duration of at least 1, 2, 3, 4, 5, 6, 7, 8, 9, 10, 11 or 12 months, or indefinitely.

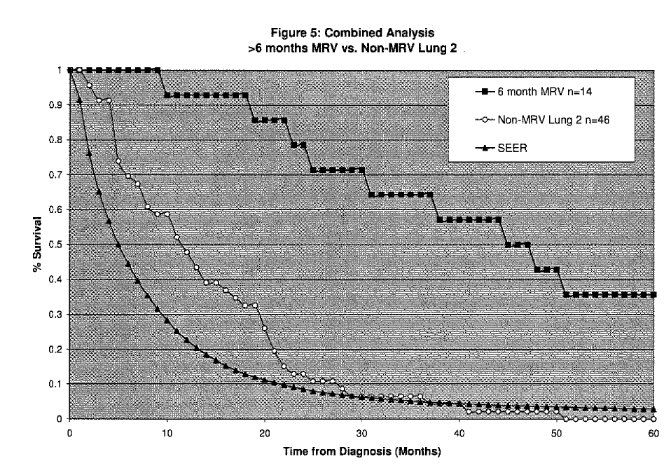

When the analysis of patient data is restricted to patients who were treated with MRV for at least 6 months, the survival curve is truly remarkable, as illustrated in FIG. 5. More than 60% of patients were alive at 3 years, more than 10 times the survival in both the non-MRV group and the SEER registry. 36% (5 of 14 patients) of patients who were treated with MRV for at least 6 months were alive 5 years after diagnosis, compared with only 1.2% in the SEER database and 0% in the non-MRV group. These remarkable results, in the context of a cancer diagnosis that is considered terminal, are extremely promising and surprising.

Restricting analysis to those patients who were treated with MRV for a minimum period of time (e.g., 6 months) introduces a bias in favour of the MRV group, since MRV patients who survived for less than that period of time are excluded from the group (including those who died before they could complete the 6 months of treatment). A detailed statistical analysis of this bias, with compensatory exclusion of short-term survivors in both the non-MRV and SEER groups, demonstrates that this bias played a very minor role in the truly remarkable survival advantage of patients who were treated with the MRV for at least 6 months.

Metastasis to the Lung

One aspect of the invention involves the treatment of primary lung cancers or metastasis to the lung with antigenic compositions that comprise antigenic determinants of microbial pathogens that cause lung infections, such as exogenous pathogens or pathogens that are members of the endogenous flora of the respiratory system. For example, antigenic determinants of the most common endogenous pathogenic bacterial species (see Table 5) may be used to treat primary and metastatic cancers situated in the lung: Streptococcus pneumoniae, Moraxella catarrhalis, Mycoplasma pneumoniae, Klebsiella pneumoniae, Haemophilus influenza, Staphylococcus aureus. Similarly, common viral pathogens from Table 5 may be selected for use in some embodiments. Alternatively, a more exhaustive list of endogenous pathogens may be selected from Table 1 based on the pathogenicity information provided in Table 2. In further alternative embodiments, viral pathogens listed in Table 4 may be used. And in further alternative embodiments, exogenous bacterial pathogens from Table 3 may be used in formulating antigenic compositions of the invention, i.e. selected from the group consisting of: Achromobacter spp., Actinomadura spp., Alcaligenes spp., Anaplasma spp., Bacillus anthracis, other Bacillus spp., Balneatrix spp., Bartonella henselae, Bergeyella zoohelcum, Bordetella holmesii, Bordetella parapertussis, Bordetella pertussis, Borrelia burgdorferi, Borrelia recurrentis, Brucella spp., Burkholderia gladioli, Burkholderia mallei, Burkholderia pseudomallei, Campylobacter fetus, Capnoctyophaga canimorsus, Capnoctyophaga cynodegmi, Chlamydia pneumoniae, Chlamydia psittaci, Chlamydophila pneumoniae, Chromobacterium violaceum, Chlamydophila psittaci, Chryseobacterium spp., Corynebacterium pseudotuberculosis, Coxiella burnetii, Francisella tularensis, Gordonia spp., Legionella spp., Leptospirosis spp., Mycobacterium avium, Mycobacterium kansasii, Mycobacterium tuberculosis, other Mycobacterium spp., Nocardia spp., Orientia tsutsugamushi, Pandoraea spp., Pseudomonas aeruginosa, other Pseudomonas spp., Rhodococcus spp., Rickettsia conorii, Rickettsia prowazekii, Rickettsia rickettsiae, Rickettsia typhi. For example, the MRV composition may be used to treat primary lung cancer or lung metastases, as illustrated in the cumulative data presented here, and in a number of the case reports.

Breast Cancer with Metastasis to the Bone or Lung

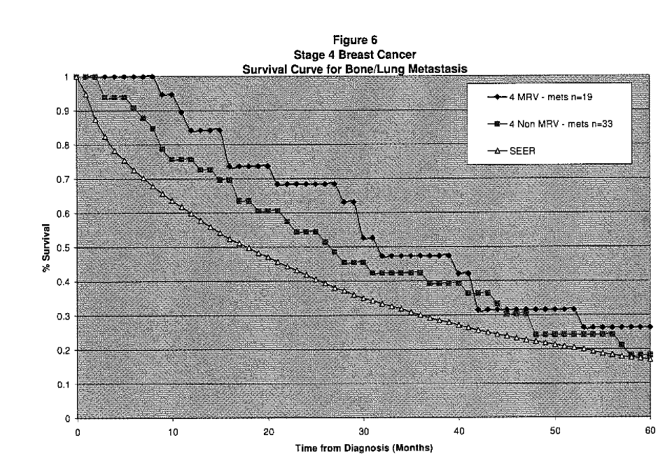

The most common cause of both breast infection and bone infection is Staphylococcus aureus. Accordingly, in one aspect of the invention, an antigenic composition comprising antigenic determinants of S. aureus may be used to treat breast cancer with metastases to the bone. The remarkable case of Patient R (PtR), set out below in the Case Reports, illustrates the efficacy of this approach to treating breast cancer. As illustrated in FIG. 6, in a cumulative series of 52 patients survival of breast cancer patients with metastases to bone and/or lung treated with MRV (n=19) is better than the survival of patients not treated with the MRV vaccine (n=33):

| | | % survival MRV patients | % survival non-MRV patients |
|---|---|---|---|
| 10 | months | 95% | 76% |
| 20 | months | 74% | 61% |
| 5 | years | 26% | 18% |

Metastases to the Bone

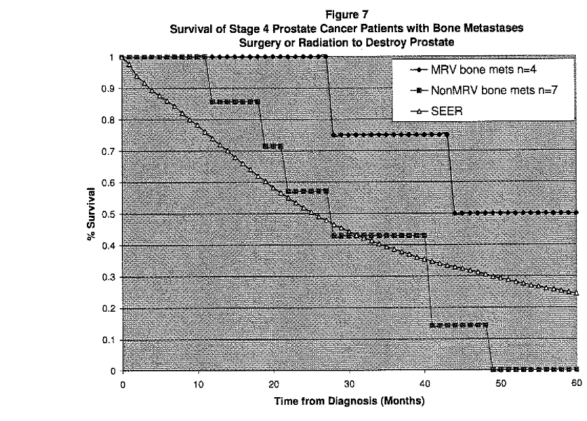

One of the most common sites for metastases in prostate cancer is bone. In one aspect of the invention, the MRV composition, which contains antigenic determinants of S. aureus, may be used for the treatment of metastases to the bone, for example in patients who have, or who have had, a primary prostate cancer. The graph of FIG. 7 is a comparison of survival of a cumulative series of metastatic prostate cancer patients who had surgery or radiation to destroy their prostate gland (and thus, the primary tumour) and who had detectable cancer limited to bone metastases. As illustrated, the survival of patients treated with MRV (n=4) is substantially better than that of patients not treated with MRV (n=7):

| | % survival MRV patients | % survival non-MRV patients |
|---|---|---|
| 2 years | 100% | 57% |
| 3 years | 75% | 43% |
| 5 years | 50% | 0% |

In accordance with the foregoing results, one aspect of the invention involves the treatment of primary bone cancers and metastasis to the bone with antigenic compositions that comprise antigentic determinants of microbial pathogens that may cause bone infections, such as exogenous pathogens or pathogens that are members of the endogenous flora of the skin, mouth or colon. For example, antigenic determinants of the following microbial species may be used to treat primary and metastatic cancers situated in the bone: Staphylococcus aureus, coagulase-negative staphylococci, Streptococcus pyogenes, Streptococcus pneumoniae, Streptococcus agalactiae, other streptococci spp., Escherichia coli, Pseudomonas spp., Enterobacter spp., Proteus spp., Serratia spp., parvovirus B19, rubella, hepatitis B.

Cancer Situated in the Colon

The PVF composition has been shown to improve the survival of colon cancer patients, as illustrated by a comparison of the following four colon cancer patient groups:
1. Stage 4 colon cancer patients who were treated with MRV.
2. Stage 4 colon cancer patients who were not treated with a vaccine.
3. Stage 4 colon cancer patients who were treated with PVF vaccine.
1. Stage 4 colon cancer patients from the SEER (Surveillance, Epidemiology and End Results) database.

This example illustrates that patients with colon cancer treated with PVF have substantially improved survival (PVF contains E. coli, the most common cause of colon infection).

Patients qualified for the first two groups of this study if they presented with stage 4 colon cancer. Patients were excluded from this analysis for the following reasons:
incorrect diagnosis
incorrect stage
missing essential data (e.g., date of death)
no chart
chart did not reach us in time for the data analysis.

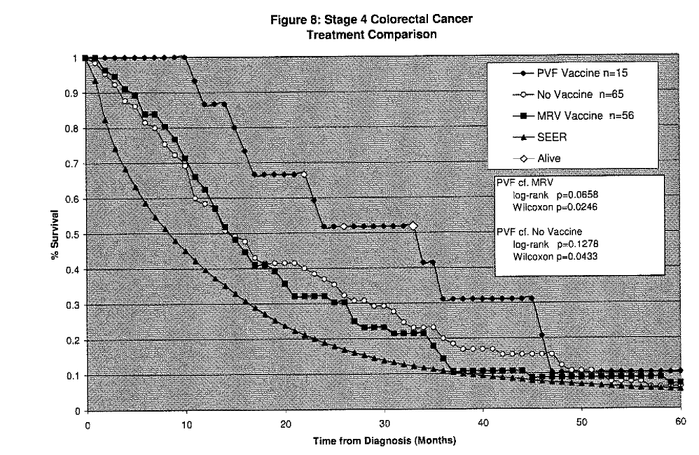

The patient group included a total of 136 stage 4 colon cancer patients: 15 who took the PVF vaccine, 56 who took the MRV vaccine, and 65 who did not take a vaccine. Results are illustrated in FIG. 8, as follows:

| | SEER | no vaccine | MRV | PVF |
|---|---|---|---|---|
| median survival: | 8.4 mo. | 15.1 mo. | 15.0 mo. | 33.6 mo. |
| at 10 months | 45% | 69% | 71% | 100% |
| at 20 months | 24% | 42% | 36% | 67% |
| at 30 months | 14% | 29% | 23% | 52% |
| at 5 years | 5% | 6% | 7% | 10% |

The median survival of patients with stage 4 colon cancer treated with PVF was more than double that of patients treated with MRV or patients not treated with a vaccine, and four times that of the SEER registry. All 15 patients treated with PVF were still alive 10 months after diagnosis, compared to only 71% for the MRV group, 69% for the no-vaccine group and only 45% for the SEER registry. Survival at 30 months for the PVF group was double that of both the MRV group and the no-vaccine group and almost 4 times that of the SEER registry.

The wilcoxon test shows a statistically significant survival difference between patients treated with PVF vaccine and both the MRV group (p=0.0246) and the no vaccine group (p=0.0433). This is remarkable considering the small size of the PVF group (n=15), indicative of substantial therapeutic effect. As evidenced by these results, the PVF composition is an effective treatment for colon cancer, and contains E. coli, the most common cause of colon and abdominal infection.

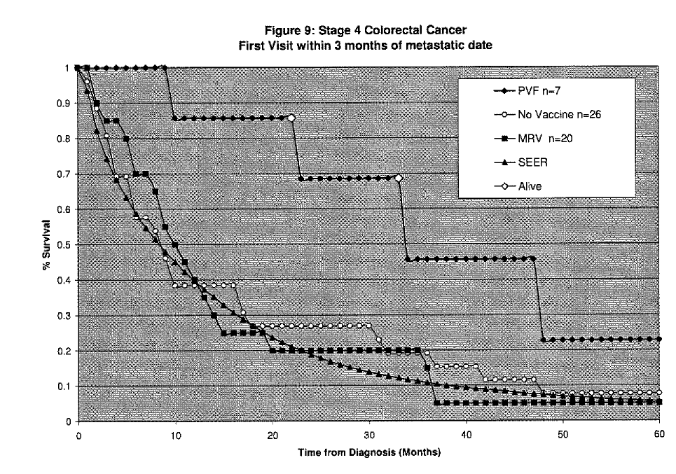

Survival of those patients who presented for immunological treatment in accordance with the invention within 3 months of diagnosis (i.e., excluding those patients who were long-term survivors before presenting for treatment) has also been analyzed. The results of this analysis are presented in FIG. 9. As illustrated, the ‘MRV’ and ‘No Vaccine’ survival curves in FIG. 9 are shifted substantially to the left (indicating that a selection bias towards ‘long-term’ survivors may have shifted these curves to the right in FIG. 8), whereas, remarkably, the PVF curve in FIG. 9 is actually further to the right than the curve in FIG. 8, indicating that earlier treatment with PVF (i.e., within 3 months of diagnosis) more than outweighed any long-term survivor bias excluded in FIG. 9. This analysis provides compelling evidence that the benefit of PVF treatment for stage 4 colon cancer may be even greater than that illustrated in FIG. 8, and that the earlier the treatment with the compositions of the invention is begun following diagnosis, the greater the benefit.

In accordance with the foregoing results, one aspect of the invention involves the treatment of colon cancers with antigenic compositions that comprise antigentic determinants of microbial pathogens that may cause colon infections, such as pathogens that are members of the endogenous flora of the colon or exogenous pathogens. For example, antigenic determinants of the following microbial species may be used to treat primary and metastatic cancers situated in the colon: Escherichia coli, Clostridium difficile, Bacteroides fragilis, Bacteroides vulgatus, Bacteroides thetaiotaomicron, Clostridium perfringens, Salmonella enteriditis, Yersinia enterocolitica, Shigella flexneri, adenoviruses, astroviruses, caliciviruses, noroviruses, rotaviruses, or cytomegalovirus. For example, cancers situated in the colon may be treated with the PVF composition, as illustrated below.

### EXAMPLE 2

### Case Reports

These case reports are indicative of the patients that make up the patient populations reflected in the foregoing cumulative studies, as well as illustrating additional aspects of the invention.

MRV for Cancer of the Lung with and without Anti-Inflammatories

Patient A (PtA): In September year 0, PtA developed right upper chest pain with an associated wheeze. These symptoms persisted and in January, year 1, she had a chest x-ray that revealed a large 7 cm×8 cm mass in the apex of the right lung. A fine needle aspiration was positive for non-small cell lung cancer. On January 27, year 1, an MRI showed invasion of the subclavian arteries, making surgical resection impossible and thus, PtA was diagnosed with stage 3B inoperable terminal lung cancer. She underwent a short course of palliative radiation and declined chemotherapy. She was told that she had terminal cancer with a 3 to 6 months life expectancy.

On April 29, year 1, PtA began therapy with MRV vaccine three times per week. On that same date she also began treatment with the non-steroidal anti-inflammatory agent (NSAID) indomethicin 50 mg four times per day and a regime of antioxidant supplements and vitamin D. 18 months later, by October, year 2, the tumour had markedly reduced in size to 3 cm in diameter and, by May 19, year 5, four years after starting treatment with the combined regime of MRV vaccine, indomethicin, antioxidants vitamins and vitamin D, only residual scarring remained. PtA continued treatment with this combination of MRV vaccine and adjuvant anti-inflammatory therapies for more than 4 years until the end of May, year 5 at which time there was no evidence of residual cancer, in spite of a diagnosis of terminal inoperable lung cancer more than 4 years previously. More than 12.5 years since diagnosis with terminal lung cancer, PtA continues to feel well with no evidence of residual cancer.

The concomitant use of anti-inflammatory agents, such as antioxidants, vitamin D and indomethicin, in conjunction with targeted MRV therapy, was associated with substantially improved survival, which was greater than that of otherwise similar cases, in which these adjuvant anti-inflammatory modalities were not used in conjunction with the compositions of the invention. For example, Patient B, an otherwise similar case in which anti-inflammatories were not administered, was diagnosed with inoperable stage 3B non-small cell lung cancer, which was fatal within 3 months of diagnosis. These cases provide evidence of a synergistic effect between the antigenic compositions of the invention and anti-inflammatory treatments.

MRV for Cancer of the Lung with and without Anti-Inflammatories

Patient C (PtC): In the spring of year 0, PtC began having pain in his right upper chest area. This pain persisted and on October 5, year 0 he had a chest x-ray that revealed a large 12 cm×11 cm mass occupying virtually the entire right upper lobe. A fine needle aspiration was positive for poorly differentiated non-small cell lung cancer. Exploratory thoracotomy was performed on December 7, year 0, which revealed tumour invasion of the chest wall and superior vena cava and therefore, PtC's tumour was inoperable (i.e., stage 3B). PtC underwent a short course of palliative radiation and declined chemotherapy. He was told that he had terminal cancer with a 3 to 6 months life expectancy. By January 27, year 1, the rapidly growing tumour had increased in size to 14 cm×11.5 cm.

On February 9, year 1, PtC began treatment with indomethicin 50 mg four times per day, antioxidant vitamins, and vitamin D. Three weeks later, on March 1, year 1, PtC began treatment with MRV vaccine three times per week. By June, year 1, PtC was feeling well and was running 8 km 3-4 times per week. On June 4, year 1, a chest x-ray revealed that the tumour had reduced in size to 11 cm diameter. PtC continued to feel very well, leading a full and active life with return to full employment and continued full physical activity. PtC continued treatment with a combination of the MRV vaccine and adjuvant anti-inflammatory therapies (indomethicin, antioxidants and vitamin D) for more than 16 months until July 24, year 2, at which time indomethicin treatment was discontinued (as a result of decreased kidney function, a known potential side-effect of long-term indomethicin use). 6 months later, in December, year 2, after 22 months of targeted vaccine therapy, MRV treatment was discontinued (since MRV was no longer available past that date). PtC continued to feel well until June, year 6, at which time he was diagnosed with a recurrence of cancer in both lungs, which lead to his death on May 26, year 7, more than 6.5 years after he was diagnosed with terminal lung cancer and told he had 3-6 months to live.

In this case, the use of adjuvant anti-inflammatory agents, including antioxidants, vitamin D and indomethicin, used in conjunction with targeted MRV therapy for more than 16 months, was associated with substantially improved survival in the face of a diagnosis that is usually fatal within 1 year, which was greater than that of an otherwise similar case, Patient D, in which these adjuvant anti-inflammatory modalities were not used in conjunction with the compositions of the invention, and an inoperable lung cancer was fatal within 8 months of diagnosis. These cases provide evidence of a synergistic effect between the antigenic compositions of the invention and anti-inflammatory treatments.

PVF for Cancer of the Colon with Metastases to the Liver and Lung, and without Anti-Inflammatories

Patient E (PtE): PtE had a surgical resection of colon cancer on June 17, year 0, followed by chemotherapy. On August 15, year 0, he was diagnosed stage 4 cancer with metastases to the liver and lungs, a diagnosis with a very poor prognosis. On October 20, year 0, PtE began treatment with an antioxidant and vitamin D regime and, on Dec 10, year 0, he began treatment with the PVF composition three times per week, which he has continued in combination with the antioxidants and vitamin D. In September, year 1, he began treatment with Celebrex 100 mg twice per day. In spite of a very poor initial prognosis, PtE is still alive more than 3 years after diagnosis with terminal metastatic colon cancer.

In contrast to PtE, of the 15 patients diagnosed with stage 4 colon cancer and treated with PVF, the patient with the shortest survival, Patient F, was not treated with anti-inflammatories. These cases provide compelling evidence that anti-inflammatory modalities (i.e., Celebrex, anti-oxidants and vitamin D) taken in conjunction with targeted PVF therapy has a synergistic effect, contributing to PtE's prolonged survival, which was greater than that of otherwise similar cases in which these adjuvant anti-inflammatory modalities were not used in conjunction with the compositions of the invention.

PVF for Cancer of the Colon with Metastases to Lung, with Anti-Inflammatories

Patient G (PtG): PtG developed rectal bleeding in May, year 0, and was diagnosed with colon cancer. He underwent surgery, chemotherapy and radiation, but developed metastases to his lungs (stage 4 cancer) on 16 August, year 1, a terminal diagnosis with a poor prognosis. He had begun a regime of antioxidant vitamins and vitamin D in June, year 0, and, on September 23, year 1, he began taking the NSAID Celebrex 100 mg twice per day. In March, year 3, he began PVF vaccine three times per week, which he continued till April, year 4 at which time he developed brain metastases, which lead to his death on June 2, year 4, almost 3 years after a diagnosis of stage 4 terminal colon cancer. PtG lived substantially longer than would normally be expected with a diagnosis of stage 4 colon cancer. In this context, the invention provides for the use of anti-inflammatory modalities in conjunction with immunogenic compositions, such as PVF, for synergistic effect.

PVF and Anti-Inflammatories for Cancer of the Colon with Metastases to the Liver and Lungs

Patient H (PtH): PtH was diagnosed with colon cancer with metastases to the liver and lungs on February 13: year 0. On January 11, year 1, he was prescribed an antioxidant and vitamin D regime. However, in March, year 1, he entered a chemotherapy research study and discontinued these supplements at that time at the request of the study coordinators. He was not treated with any NSAIDs. On May 12, year 1, he began treatment with PVF, which he took three times per week until his death just 2.5 months later. When contrasted to similar cases that involved the use of anti-inflammatories, this case illustrates a lack of a synergistic effect of adjuvant anti-inflammatory modalities.

In summary, in cases of stage 4 colon cancer treated with targeted PVF vaccine therapy, the use of adjuvant anti-inflammatory agents, including antioxidants, vitamin D and Celebrex, used in conjunction with targeted antigenic activation therapy, was associated with substantially improved survival, much greater than that of the two cases in which these adjuvant anti-inflammatory modalities were not used in conjunction with the vaccine, providing evidence suggestive of a synergistic effect.

PVF with and without Anti-Inflammatories for Cancer of the Pancreas with Metastases to the Lungs

Patient I (Ptl): Ptl was diagnosed with pancreatic cancer in August, year 1, at which time he had surgery to remove his pancreas (i.e., Whipple's procedure). However, in July year 2, he developed metastases to the lungs bilaterally and in February year 4 he developed recurrence of cancer in the pancreatic area with abdominal and liver metastases. This is a terminal diagnosis with a very poor prognosis. Ptl began a regime of antioxidant vitamins, vitamin D, large doses of turmeric (curcumin), fish oil (9 gm per day), resveratrol and green tea (equivalent of 36 cups per day) on September 27, year 2, all of which are anti-inflammatory modalities, all of which he continues to take. In March year 3 he began treatment with Celebrex 100 mg twice per day, which he took for more than 20 months. Ptl began treatment with PVF three times per week in May year 4, which he has continued to use regularly. Ptl is alive more than 4 years after a diagnosis of terminal metastatic pancreatic cancer, a remarkably prolonged survival in the context of a diagnosis that has an extremely poor prognosis. This case provides evidence that high doses of multiple anti-inflammatory modalities (i.e., Celebrex, antioxidants, vitamin D, turmeric, fish oil, resveratrol, green tea) taken in conjunction with the PVF compositions, resulted in a synergistic effect which contributed to Ptl's remarkable survival in the context of a diagnosis (i.e., metatstatic pancreatic cancer) that is usually fatal within 6 months.

Patient J (PtJ) had an essentially identical diagnoses to Ptl (i.e., pancreatic cancer with metastases to abdominal lymph nodes, lungs and liver). PtJ, who did not take any other anti-inflammatories along with the PVF vaccine except antioxidants, died within 4 months of diagnosis, whereas Ptl, who took large doses of numerous other anti-inflammatories modalities (i.e., Celebrex, turmeric, fish oil, resveratrol and green tea) in conjunction with PVF vaccine, is still alive more than 4 years after diagnosis. These cases provide evidence of a synergistic effect of high dose multiple anti-inflammatory modalities and targeted vaccine therapy.

MRV for Cancer of the Breast with Metastases to the Cervical Spine

Patient K (PtK): In March, year 0, PtK developed neck and back pain, which persisted. On July 28, year 0, she was diagnosed with stage 4 breast cancer with metastases to the cervical spine, an incurable diagnosis. She underwent surgery to remove two breast lumps (axillary lymph nodes positive) and palliative radiation to the metastases in her spine. On January 18, year 1, PtK began treatment with doses of antioxidants and vitamin D, as well as the NSAID indomethicin 50 mg four times per day. Three days later, on January 21, year 1, she began treatment with the MRV composition. Although there was no documentation of the exact length of time that treatment with this combination of MRV/indomethicin/antioxidant/vitamin D was continued, the patient was given sufficient vaccine (20 ml) for approximately 2 years of treatment at the usual dose and frequency (i.e., three times per week) and PtK states that she completed the recommended treatment course at home. Remarkably, PtK is still alive, 13 years after diagnosis with stage 4 metastatic breast cancer with metastases to bone.

In contrast to Patient K, Patient L (PtL) was diagnosed with breast cancer with metastases to bone on October 11, year 0. She was not prescribed an NSAID or other anti-inflammatories. PtL began treatment with MRV on February 27, year 1. She died 9 months later on November 4, year 1, just over one year after diagnosis with stage 4 breast cancer with metastases to bone. The contrast between the otherwise similar cases of PtK and PtL illustrates the potential for synergistic treatment with anti-inflammatories and the antigenic compositions of the invention.

MRV with and without Anti-Inflammatories for Cancer of the Breast with Metastases to the Bone

Patient M (PtM): PtM was diagnosed with breast cancer in year 0, and then diagnosed with stage 4 cancer with metastases to bone on June 15, year 3. She began on the NSAID Naprosyn 250 mg twice per day on an ongoing basis for pain relief and, in October, year 6, she began doses of antioxidants and vitamin D. On January 15, year 7, she began treatment with MRV vaccine in combination with these anti-inflammatory therapies (i.e., Naprosyn, antioxidants and vitamin D). PtM lived for more than 9 years after being first diagnosed with stage 4 metastatic breast cancer with metastases to bone, an unusually long survival considering the usual poor prognosis associated with this diagnosis.

In contrast to PtM, Patient N (PtN): PtN was diagnosed with stage 4 cancer with metastases to bone on April 8, year 0. She began doses of antioxidants and vitamin D on April 24, year 0. However, prior to starting MRV, she was prescribed the blood thinner warfarin, limiting supplementation with vitamin E and vitamin C, two important antioxidants that can lead to potential complications if used in conjunction with warfarin. In addition, NSAIDs could not be prescribed in this case since they are contraindicated with warfarin use. On June 2, year 1 PtN began treatment with MRV. She died 14 months later in August, year 2. In this context, it is possible that the use of targeted vaccine therapy without the synergistic effect of adjuvant anti-inflammatories (i.e., NSAID, vitamin E and therapeutic doses of vitamin C) limited its potential benefit.

In summary, in the cases of stage 4 breast cancer with metastases to the bone treated with targeted MRV therapy detailed above, the use of adjuvant anti-inflammatory agents in conjunction with MRV was associated with substantially improved survival, much greater than that of the two cases in which these adjuvant anti-inflammatory modalities were not used in conjunction with the vaccine, providing evidence suggestive of a synergistic effect.

MRV for Metastases to the Lungs

Patient O (PtO) was diagnosed in June, year 0 with kidney cancer with metastases to the lungs bilaterally and to the bone (left femur). This is generally considered to be an incurable terminal diagnosis with a poor prognosis. He began treatment with the MRV on August 10, year 0 and continued regular treatment (three times per week) for 16 months (after which MRV was no longer available). In September, year 0, he began 7 months of treatment with an experimental drug, pegylated interferon alpha-2a. His left femur was ‘pinned’ due to the risk of fracture as a result of the metastasis but, due to surgical complications, amputation of the left leg below the mid-thigh was required. In September, year 2, his cancerous right kidney was removed. In October, year 2, a PET scan found no evidence of cancer in the lungs and no further evidence of bone metastases. PtO is alive with no evidence of cancer in his lungs, more than 7 years after a diagnosis of bilateral pulmonary metastases, a remarkable result.

MRV for Metastases to the Bone

Patient P (PtP) was diagnosed with kidney cancer in July, year 0, and underwent excision of this right kidney. In December, year 4, he developed metastases to the bone (femurs bilaterally) and lung (bilaterally). PtP declined conventional treatment and began treatment with MRV in April, year 5, which he continued regularly, three times per week, for 18 months. PtP's health improved and he returned to normal daily activities. X-rays and imaging of the chest and femurs showed no progression, with stable disease in the lungs and femurs during the 18 months that PtP was on MRV treatment.

MRV for Metastases to the Lungs

Patient Q (PtQ) was diagnosed with colon cancer with probable metastases to the lungs in June, year 0. At that time, the primary colon tumour was fully excised, leaving only several lung metastases. PtQ started treatment with MRV on Dec. 11, year 0 which she continued three times per week for 4 months. On April 19, year 1 after 6 months treatment with chemotherapy, she had surgery to excise the only visible lung lesion remaining, which was confirmed to be a metastatic lesion. A diagnosis of colon cancer with lung metastases has a poor prognosis, even in the context of chemotherapy followed by surgery to excise visible metastases. In spite of her original poor prognosis, PtQ remains in excellent health, with no evidence of cancer more than 8 years after her initial diagnosis with metastases to the lung and treatment with MRV.

S. aureus Antigens for Breast Cancer with Metastasis to the Bone

Patient R (PtR): In May, year 0, PtR was diagnosed with breast cancer with metastases to her sternum, femur and cervical spine, an incurable cancer with a poor prognosis. She was treated with radiation and Tamoxefen. In May, year 4, she developed an additional area of metastasis in her lumbar spine and she began on treatment with Megace. In November, year 4, she began treatment with a vaccine containing only S. aureus (Staphage Lystate vaccine), the optimal targeted vaccine formulation for the treatment of breast and bone cancer. She continued regular therapy with this vaccine for 5 years. In spite of a diagnosis of metastatic breast cancer with multiple bone metastases, PtR survived for more than 17 years, a remarkable survival in the context of incurable metastatic breast cancer and a testament to the promise of targeted vaccine therapy for the treatment of breast cancer.

MRV for Multiple Myeloma

Patient S (PtS) was diagnosed with multiple myeloma (stage 3A) in the fall of year 0, with multiple lesions on bone scan, including skull, humeri and pelvis. He was treated with standard chemotherapy (melphalan and prednisone) for 6 months. However, in December year 3, he developed a pathological fracture of his right femur as a result of his disease, which required pinning a local radiation. On April 28, year 4, PtS began treatment with MRV, which he continued for more than 13 years until this vaccine was no longer available in December year 17. Remarkably, PtS was still alive 23 years after being diagnosed with multiple myeloma, a truly extraordinary outcome considering his ‘terminal’ diagnosis.

PVF for Colon Cancer with Metastases of the Liver and Abdominal Lymph Nodes

Patient T (PtT) was diagnosed with colon cancer and was treated with excision of the primary tumour (and subsequent chemotherapy) in September year 0. Ten months later, she developed a liver metastasis, which was surgically excised in July year 1. PtT remained well until June year 7, when she was diagnosed with recurrent disease—an inoperable mass of abdominal lymph nodes in close proximity to the aorta and spine, obstructing her left ureter, requiring insertion of a nephrostomy tube. PtT was considered terminal and treated with palliative radiation in October year 7. She began treatment with PVF on November 17, year 7, which she has continued every second day since. PtT continues to feel very well and is leading a very full and active life, almost 3 years since being diagnosed with inoperable terminal cancer.

MRV for Metastasis to the Skin and Perineum

Patient U (PtU) was diagnosed with colon cancer and was treated with excision of the primary tumour in November year 0. He was diagnosed with stage 4 cancer in July year 2 with metastases to the perineum (i.e., peri-anal/genital soft tissue area) and skin. He had further surgery to remove as much of the cancer as possible in the perineum (cancer extended past surgical margins) with follow-up radiation and chemotherapy. The only known cancer sites remaining were in the skin and perineum. PtU started treatment with MRV on May 25, year 3, which he continued three times per week for 5 months. In spite of his original poor prognosis, PtU is in excellent health almost 8 years after his diagnosis with stage 4 cancer with metastases to the perineum and skin.

PVF for Metastases to the Peritoneum

Patient V (PtV) was diagnosed with breast cancer in May, year 0, at which time she had a masectomy with adjuvant chemotherapy. In January, year 12, she developed abdominal pain and ascites and was diagnosed with peritoneal metastases, a diagnosis with a poor prognosis. On August 5, year 12, PtV began treatment with PVF, which she continued regularly for 1 year. Her tumour markers and ascites decreased and, in August year 13, after one year of PVF treatment, she had abdominal surgery for an unrelated medical condition, at which time the surgeon could not find any evidence of the previous peritoneal cancer. PtV discontinued use of the vaccine. PtV is alive, 3 years and 9 months after being diagnosed with terminal peritoneal metastases.

PVF for Ovarian Cancer

Patient W (PtW) was diagnosed with stage 3B poorly differentiated ovarian cancer in the fall of year 0. She had surgery in November, year 0, with removal of the left ovary, but the cancer could not be completely excised and thus, she was at extreme risk for recurrence. She had a full course of post-operative chemotherapy. However, in year 2 her tumour markers began to rise and in January year 3 she was diagnosed with a recurrence in her right ovary area. She surgery to remove this right ovarian mass in February year 3, but again the cancer could not be completely excised and she had follow-up chemotherapy. However, once again in December year 3 she developed a further recurrence in the pelvic area and retroperitoneal lymphadenopathy. She began treatment with PVF vaccine on January 5, year 4, which she continued for 6 months. Her tumour markers, which had risen to 2600, fell to the 300 range. PtW is alive and feeling very well, 2 years and 9 months after being diagnosed with recurrent ovarian cancer. Of note is the fall in her tumour markers following PVF treatment.

MRV for Follicular Non-Hodgkin's Lymphoma

Patient Y (PtY): was diagnosed with stage 4A Follicular Non-Hodgkin's lymphoma, with extensive marked lymphadenopathy (i.e., enlarged lymph glands). He declined all conventional treatment. PtY began treatment with the MRV composition, as well as the multiple vitamin/supplement regimes, healthful diet and other immune enhancement treatments. He continued regular use of this vaccine for more than 3 years, at which time his lymph glands had begun to greatly reduce in size and he was feeling well. This resolution of lymphadenopathy continued, and imaging showed almost complete resolution of previous extensive lymphadenopathy. PtY was feeling well and there was no lymphadenopathy palpable: a clearly remarkable recovery. Five years after his initial diagnosis with Stage 4A Follicular Non-Hodgkin's lymphoma, PtY had no evidence of recurrence and was leading an active and healthy life. Treatment with the MRV vaccine resulted in complete remission of his stage 4A follicular non-Hodgkins' lymphoma.

PVF for Colon Cancer with Metastases to the Liver

Patient Z (PtZ) was diagnosed with metastatic spread of previously treated colon cancer, with a metastasis to the liver and probable other metastases to both kidneys. The liver metastasis was excised. The prognosis for this stage (i.e., stage 4) of colon cancer is poor and the benefit of further conventional treatment (i.e., chemotherapy) is limited. PtZ declined chemotherapy initially. Three months after diagnosis with metastatic colon cancer, PtZ began treatment with Polyvaccinum Forte (PVF), as well as a multiple vitamin/supplement regime and healthful diet. He continued regular use of this vaccine and the vitamin and supplement regime, and began chemotherapy. Although the overall course of his disease has been slowly progressive, with development of lung metastases and recurrence of liver metastases, 28 months after his initial diagnosis of metastatic disease, his weight was stable and his energy levels were good. Three years (36 months) after diagnosis of stage 4 colon cancer, PtZ was feeling well except for nausea and mild weight loss related to chemotherapy.

PVF for Colon Cancer with Metastases to the Liver, Porta Hepatic Lymph Nodes and Lung

Patient BB (PtBB) was diagnosed with metastatic colon cancer with metastases to the liver, porta hepatic lymph nodes and lungs. The prognosis for this stage (i.e., stage 4) of colon cancer is very poor (i.e., ‘terminal’ cancer) and the benefit of conventional treatment (i.e., chemotherapy) is limited. PtBB began chemotherapy, but discontinued treatment approximately 5 months after his diagnosis due to side effects, at which time he began treatment with Polyvaccinum Forte (containing E. coli) every second day as well as a multiple vitamin/supplement regime and a healthy diet. PtBB's subsequent CT Scans demonstrated necrotic porta hepatic lymph nodes unchanged in size from the time of his diagnosis and no change in size of the lung metastases, although the two liver metastases grew moderately in size (3.4 cm to 4.5 cm and 1.2 cm to 3.0 cm). In spite of the very poor prognosis, PtBB continued to feel quite well almost one year after a diagnosis of terminal cancer.

### EXAMPLE 3

### Microbial Pathogens

In alternative aspects, the invention utilizes microbial antigens, such as bacterial or viral antigens, to formulate antigenic compositions, where the microbial species is selected on the basis of the tissue or organ within which the microbe is known to cause infections. Bacterial resident flora account for the vast majority of infectious episodes of most animals, including humans. Resident flora can for example infect through primary attachment, or attachment and invasion following mucosa damage, resulting for example from vascular, trauma, chemical insult, or damage resulting from primary infection.

For microbial pathogens, virulence and infection potential is a combination of the ability of the microbe to adhere, to produce enzymes, to survive immunoproducts (complement, antibody) and to survive the microbiocidal activity of macrophage and neutrophils. Some bacteria, including endogenous bacteria, may be sufficiently virulent as to cause monomicrobial infections, others are more effective with the synergy of polymicrobial infection. In general, it is often not possible to be precise about the specific role of individual microbes within the milieu of mixed infection. Bacteria successful at intracellular survival within macrophages are more commonly associated with chronic infection, as are bacteria with slow growth cycles. Accordingly, in some embodiments, the invention utilizes microbial species that are involved in acute infection.

In various aspects of the invention, in addition to exogenous mibrobes, bacteria that are members of the endogenous flora of a particular region may be used to formulate antigenic compositions of the invention. The rows of Table 1 list a number of bacterial species, together with the biological regions in which each species may form a part of the endogenous flora. For example, Abiotrophia spp. are typically members of the endogenous flora of respiratory tissues and the mouth.

TABLE 1
Human Bacterial Normal Flora (Endogenous Bacterial Human Pathogens)

| | | | | Duodenum/ | | | GU | | |
|---|---|---|---|---|---|---|---|---|---|
| Bacterial species | Respiratory | Mouth | Stomach | Jejunum | Ileum | Colon | System | Genital | Skin |
| CFL/mL | | 10\hat{ }5 | 10\hat{ }2 | 10\hat{ }5 | 10\hat{ }8 | 10\hat{ }11 | | | |
| Abiotrophia spp | + | + | | | | | | | |
| Acholeplasma | + | + | | | | | | | |
| laidlawii | | | | | | | | | |
| Acidaminococcus | + | + | | + | + | + | | | |
| fermentans | | | | | | | | | |
| Acinetobacter spp. | + | + | | + | + | + | + | + | + |
| Actinobacillus spp. | + | + | | | | | | | |
| Actinobaculum spp. | + | + | | + | + | + | | | |
| Actinomyces spp. | + | + | | + | + | + | + | + | |
| Aerococcus | | | | | | | + | | |
| christensenii | | | | | | | | | |
| Aerococcus viridans | | | | | | | | | + |
| Aerococcus urinae | | | | | | | + | | |
| Aeromonas spp. | | | | + | + | + | | | |
| Alloiococcus otitis | | | | | | | | | + |
| Anaerorhabdus | | | | | + | + | | | |
| furcosus | | | | | | | | | |
| Anaerococcus | | | | | + | + | + | | + |
| hydrogenalis | | | | | | | | | |
| Anaerococcus | | | | | + | + | + | | |
| lactolyticus | | | | | | | | | |
| Anaerococcus | | | | | + | + | + | | |
| prevotii | | | | | | | | | |
| Arcanobacterium | + | | | | | | | | + |
| spp. | | | | | | | | | |
| Atopobium spp. | + | + | | + | + | + | | | |
| Bacillus spp. | | | | | + | + | | | + |
| Bacteroides caccae | | | | | + | + | | | |
| Bacteroides | | | | | + | + | | | |
| distasonis | | | | | | | | | |
| Bacteroides eggerthii | | | | | + | + | | | |
| Bacteroides fragilis | | | | | + | + | + | + | |
| Bacteroides merdae | | | | | + | + | | | |
| Bacteroides ovatus | | | | | + | + | | | |
| Bacteroides | | | | | + | + | | | |
| splanchnicus | | | | | | | | | |
| Bacteroides | | | | | + | + | | | |
| thetaiotaomicron | | | | | | | | | |
| Bacteroides vulgatus | | | | | + | + | | | |
| Bifidobacterium | | | | + | + | + | | | |
| adolescentis | | | | | | | | | |
| Bifidobacterium | | | | + | + | + | + | + | |
| bifidum | | | | | | | | | |
| Bifidobacterium | | | | + | + | + | + | + | |
| breve | | | | | | | | | |
| Bifidobacterium | | | | + | + | + | + | + | |
| catenulatum | | | | | | | | | |
| Bifidobacterium | + | + | | + | + | + | + | + | |
| dentium | | | | | | | | | |
| Bifidobacterium | | | | + | + | + | + | + | |
| longum | | | | | | | | | |
| Bilophila | + | + | | + | + | + | + | + | |
| wadsworthia | | | | | | | | | |
| Brevibacterium casei | | | | | | | | | + |
| Brevibacterium | | | | | | | | | + |
| epidermidis | | | | | | | | | |
| Burkholderia cepacia | + | | | + | + | + | | | |
| Butyrivibrio | | | | + | + | + | | | |
| fibrisolvens | | | | | | | | | |
| Campylobacter | + | | | + | + | + | | | |
| concisus | | | | | | | | | |
| Campylobacter | + | | | + | + | + | | | |
| curvus | | | | | | | | | |
| Campylobacter | + | | | + | + | + | | | |
| gracilis | | | | | | | | | |
| Campylobacter jejuni | | | | + | + | + | | | |
| Campylobacter | | | | + | + | + | | | |
| rectus | | | | | | | | | |
| Campylobacter | + | + | | + | + | + | | | |
| showae | | | | | | | | | |
| Campylobacter | + | + | | | | | | | |
| sputorum | | | | | | | | | |
| Capnocytophaga | + | + | | | | | | | |
| granulosum | | | | | | | | | |
| Capnocytophaga | + | + | | | | | | | |
| gingivalis | | | | | | | | | |
| Campylobacter | + | + | | | | | | | |
| haemolytica | | | | | | | | | |
| Capnocytophaga | + | + | | + | + | + | + | + | |
| ochracea | | | | | | | | | |
| Capnocytophaga | + | + | | | | | | | |
| sputigena | | | | | | | | | |
| Cardiobacterium | + | + | | | | | | | |
| hominis | | | | | | | | | |
| Cedecea spp | | | | | | + | | | |
| Centipeda periodontii | + | + | | | | | | | |
| Citrobacter freundii | | | | + | + | + | | | |
| Citrobacter koseri | | | | + | + | + | | | |
| Clostridium spp. | | | | + | + | + | | | |
| Corynebacterium | + | + | | | | | | | + |
| accolens | | | | | | | | | |
| Corynebacterium | + | + | | | | | | | + |
| afermentans | | | | | | | | | |
| Corynebacterium | | | | | | | | | + |
| amycolatum | | | | | | | | | |
| Corynebacterium | | | | | | | | | + |
| auris | | | | | | | | | |
| Corynebacterium | + | | | | | | | | + |
| diphtheriae | | | | | | | | | |
| Corynebacterium | + | | | | | | | | |
| durum | | | | | | | | | |
| Corynebacterium | | | | | | | + | | |
| glucuronolyticum | | | | | | | | | |
| Corynebacterium | | | | | | | | | + |
| jeikeium | | | | | | | | | |
| Corynebacterium | | | | | | | | | + |
| macginleyi | | | | | | | | | |
| Corynebacterium | + | | | | | | | | |
| matruchotii | | | | | | | | | |
| Corynebacterium | | | | | | | | | + |
| minutissimum | | | | | | | | | |
| Corynebacterium | + | | | | | | | | |
| propinquum | | | | | | | | | |
| Corynebacterium | + | | | | | | | | |
| pseudodiphtheriticum | | | | | | | | | |
| Corynebacterium | | | | | | | + | | |
| riegelii | | | | | | | | | |
| Corynebacterium | | | | | | | | | + |
| simulans | | | | | | | | | |
| Corynebacterium | + | | | | | | | | + |
| striatum | | | | | | | | | |
| Corynebacterium | + | | | | | | | | |
| ulcerans | | | | | | | | | |
| Corynebacterium | | | | | | | + | | + |
| urealyticum | | | | | | | | | |
| Dermabacter hominis | | | | | | | | | + |
| Dermacoccus | | | | | | | | | + |
| nishinomiyaensis | | | | | | | | | |
| Desulfomonas pigra | | | | + | + | + | | | |
| Dysgonomonas spp. | | | | + | + | + | | | |
| Eikenella corrodens | + | + | | + | + | + | | | |
| Enterobacter | | | | + | + | + | | | |
| aerogenes | | | | | | | | | |
| Enterobacter cloacae | | | | + | + | + | | | |
| Enterobacter | | | | + | + | + | | | |
| gergoviae | | | | | | | | | |
| Enterobacter | | | | + | + | + | | | |
| sakazakii | | | | | | | | | |
| Enterobacter | | | | + | + | + | | | |
| taylorae | | | | | | | | | |
| Enterococcus spp. | | | | + | + | + | | | |
| Escherichia coli | | | | + | + | + | + | + | |
| Escherichia | | | | + | + | + | | | |
| fergusonii | | | | | | | | | |
| Escherichia | | | | + | + | + | | | |
| hermannii | | | | | | | | | |
| Escherichia vulneris | | | | + | + | + | | | |
| Eubacterium spp. | + | + | | + | + | + | | | |
| Ewingella americana | + | + | | | | | | | |
| Finegoldia magnus | | | | + | + | + | + | | + |
| Fusobacterium alocis | + | + | | | | | | | |
| Fusobacterium | | | | + | + | + | + | + | |
| gonidiaformans | | | | | | | | | |
| Fusobacterium | | | | + | + | + | | | |
| mortiferum | | | | | | | | | |
| Fusobacterium | | | | + | + | + | + | + | |
| naviforme | | | | | | | | | |
| Fusobacterium | + | + | | + | + | + | | | |
| necrophorum | | | | | | | | | |
| Fusobacterium | + | + | | | | | | | |
| nucleatum | | | | | | | | | |
| Fusobacterium sulci | + | + | | | | | | | |
| Fusobacterium russii | | | | + | + | + | | | |
| Fusobacterium | | | | + | + | + | | | |
| varium | | | | | | | | | |
| Gardnerella | | | | + | + | + | + | + | |
| vaginalis | | | | | | | | | |
| Gemella haemolysans | + | + | | | | | | | |
| Gemella morbillorum | + | + | | + | + | + | | | |
| Globicatella spp. | | + | | | | + | | | |
| Granulicatella spp. | + | + | | | | | | | |
| Haemophilus spp. | + | + | | | | | | + | |
| Hafnia alvei | | | | + | + | + | | | |
| Helcococcus kunzii | | | | | | | | | + |
| Helicobacter spp. | | | | + | + | + | | | |
| Kingella spp. | + | + | | | | | | | |
| Klebsiella spp. | + | + | | + | + | + | | | |
| Kocuria spp. | | | | | | | | | + |
| Kytococcus | | | | | | | | | + |
| sedentarius | | | | | | | | | |
| Lactobacillus | + | + | + | + | + | + | + | + | |
| acidophilus | | | | | | | | | |
| Lactobacillus breve | + | + | | | | | | | |
| Lactobacillus casei | + | + | | | | | + | + | |
| Lactobacillus | | | | | | | + | + | |
| cellobiosus | | | | | | | | | |
| Lactobacillus | + | + | + | + | + | + | + | + | |
| fermentum | | | | | | | | | |
| Lactobacillus reuteri | | | + | + | + | + | | | |
| Lactobacillus | + | + | + | + | + | + | | | |
| salivarius | | | | | | | | | |
| Lactococcus spp. | | | | | | | + | + | |
| Leclercia | | | | + | + | + | | | |
| adecarboxylata | | | | | | | | | |
| Leminorella spp. | | | | + | + | + | | | |
| Leptotrichia buccalis | + | + | | | | | + | + | |
| Leuconostoc spp. | | | | | | | + | + | |
| Megasphaera | | | | + | + | + | | | |
| elsdenii | | | | | | | | | |
| Micrococcus luteus | + | + | | | | | | | + |
| Micrococcus lylae | + | + | | | | | | | + |
| Micromonas micros | + | + | | | | | | | |
| Mitsuokella | | | | + | + | + | | | |
| multiacidus | | | | | | | | | |
| Mobiluncus curisii | | | | + | + | + | | + | |
| Mobiluncus mulieris | | | | + | + | + | | + | |
| Moellerella | | | | + | + | + | | | |
| wisconsensis | | | | | | | | | |
| Moraxella | + | + | | | | | | | |
| catarrhalis | | | | | | | | | |
| other Moraxella spp. | + | + | | | | | + | | + |
| Morganella morganii | | | | + | + | + | | | |
| Mycoplasma buccale | + | + | | | | | | | |
| Mycoplasma faucium | + | | | | | | | | |
| Mycoplasma | + | + | | | | | + | | |
| fermentans | | | | | | | | | |
| Mycoplasma | + | | | | | | + | | |
| genitalium | | | | | | | | | |
| Mycoplasma hominis | + | + | | | | | + | | |
| Mycoplasma | + | + | | | | | | | |
| lipophilum | | | | | | | | | |
| Mycoplasma orale | + | + | | | | | | | |
| Mycoplasma | | | | | | | + | | |
| penetrans | | | | | | | | | |
| Mycoplasma | + | + | | | | | | | |
| pneumoniae | | | | | | | | | |
| Mycoplasma | | | | | | | + | | |
| primatum | | | | | | | | | |
| Mycoplasma | + | + | | | | | | | |
| salivarium | | | | | | | | | |
| Mycoplasma | | | | | | | + | | |
| spermatophilum | | | | | | | | | |
| Neisseria cinerea | + | | | | | | | | |
| Neisseria flavescens | + | | | | | | | | |
| Neisseria lactamica | + | | | | | | | | |
| Neisseria | + | | | | | | | + | |
| meningitidis | | | | | | | | | |
| Neisseria mucosa | + | | | | | | | | |
| Neisseria | + | | | | | | | | |
| polysaccharea | | | | | | | | | |
| Neisseria sicca | + | | | | | | | | |
| Neisseria subflava | + | | | | | | | | |
| Oligella urealytica | | | | | | | + | + | |
| Oligella urethralis | | | | | | | + | + | |
| Pantoea agglomerans | | | | + | + | + | | | |
| Pastuerella bettyae | | | | | | | + | + | |
| Pasteurella | + | + | | | | | | | |
| multocida | | | | | | | | | |
| Pediococcus spp. | | + | | | | + | | | |
| Peptococcus niger | | | | | | | + | + | + |
| Peptoniphilus | | | | + | + | + | + | + | + |
| asaccharolyticus | | | | | | | | | |
| Peptoniphilus | + | | | | | | | | |
| lacrimalis | | | | | | | | | |
| Peptostreptococcus | + | + | | + | + | + | | | |
| anaerobus | | | | | | | | | |
| Peptostreptococcus | | | | + | + | + | | | |
| productus | | | | | | | | | |
| Peptostreptococcus | | | | | | | + | + | + |
| vaginalis | | | | | | | | | |
| Porphyromonas | | + | | + | + | + | + | + | |
| asaccharolytica | | | | | | | | | |
| Porphyromonas | + | + | | + | | | | | |
| catoniae | | | | | | | | | |
| Porphyromonas | + | + | | + | | | | | |
| endodontalis | | | | | | | | | |
| Porphyromonas | + | + | | + | | | | | |
| gingivalis | | | | | | | | | |
| Prevotella bivia | | | | | | | + | + | |
| Prevotella buccae | + | + | | + | | | | | |
| Prevotella buccalis | + | + | | + | | | + | + | |
| Prevotella corporis | + | + | | + | | | | | |
| Prevotella dentalis | + | + | | + | | | | | |
| Prevotella denticola | + | + | | + | | | | | |
| Prevotella disiens | | | | | | | + | + | |
| Prevotella enoeca | + | + | | + | | | | | |
| Prevotella | + | + | | + | | | | | |
| heparinolytica | | | | | | | | | |
| Prevotella intermedia | + | + | | + | | | | | |
| Prevotella loescheii | + | + | | + | | | + | + | |
| Prevotella | + | + | | + | | | + | + | |
| melaninogenica | | | | | | | | | |
| Prevotella nigrescens | + | + | | + | | | | | |
| Prevotella oralis | + | + | | + | | | + | + | |
| Prevotella oris | + | + | | + | | | | | |
| Prevotella oulorum | + | + | | + | | | | | |
| Prevotella tannerae | + | + | | + | | | | | |
| Prevotella veroralis | + | + | | + | | | + | + | |
| Prevotella | + | + | | + | | | | | |
| zoogleoformans | | | | | | | | | |
| Propionibacterium | | | | | | | | | + |
| acnes | | | | | | | | | |
| Propionibacterium | | | | | | | | | + |
| avidum | | | | | | | | | |
| Propionibacterium | | | | | | | | | + |
| granulosum | | | | | | | | | |
| Propionibacterium | + | + | | | | | | | |
| propionicum | | | | | | | | | |
| Propionferax | | | | | | | | | + |
| innocuum | | | | | | | | | |
| Proteus mirabilis | | | | | + | + | + | | |
| Proteus penneri | | | | | + | + | + | | |
| Proteus vulgaris | | | | | + | + | + | | |
| Providencia rettgeri | | | | | + | + | | | |
| Providencia stuartii | | | | + | + | + | | | |
| Pseudomonas | | | | + | + | + | | | |
| aeruginosa | | | | | | | | | |
| Retortamonas | | | | + | + | + | | | |
| intestinalis | | | | | | | | | |
| Rothia dentocariosa | + | + | | | | | | | |
| Rothia mucilaginosa | + | + | | | | | | | |
| Ruminococcus | | | | + | + | + | | | |
| productus | | | | | | | | | |
| Selenomonas spp. | + | + | | | | | | | |
| Serratia liquefaciens | | | | | + | + | | | |
| Serratia marcescens | | | | | + | + | | | |
| Serratia odorifera | | | | | + | + | | | |
| Staphylococcus | + | + | | | | | + | + | + |
| aureus | | | | | | | | | |
| Staphylococcus | | | | | | | | | + |
| auricularis | | | | | | | | | |
| Staphylococcus | | | | | | | | | + |
| capitis | | | | | | | | | |
| Staphylococcus | | | | | | | | | + |
| caprae | | | | | | | | | |
| Staphylococcus | | | | | | | | | + |
| cohnii | | | | | | | | | |
| Staphylococcus | + | + | | | | | + | + | + |
| epidermidis | | | | | | | | | |
| Staphylococcus | | | | | | | | | + |
| haemolyticus | | | | | | | | | |
| Staphylococcus | | | | | | | | | + |
| hominis | | | | | | | | | |
| Staphylococcus | | | | | | | | | + |
| lugdunensis | | | | | | | | | |
| Staphylococcus | | | | | | | | | + |
| pasteuri | | | | | | | | | |
| Staphylococcus | | | | | | | | | + |
| saccharolyticus | | | | | | | | | |
| Staphylococcus | | | | | | | + | | + |
| saprophyticus | | | | | | | | | |
| Staphylococcus | | | | | | | | | + |
| schleiferia | | | | | | | | | |
| Staphylococcus | | | | | | | | | + |
| simulans | | | | | | | | | |
| Staphylococcus | | | | | | | | | + |
| xylosus | | | | | | | | | |
| Staphylococcus | | | | | | | | | + |
| warneri | | | | | | | | | |
| Streptococcus | | | | + | + | + | + | + | |
| agalactiae | | | | | | | | | |
| Streptococcus | + | + | | + | + | + | + | + | |
| anginosus | | | | | | | | | |
| Streptococcus bovis | | | | + | + | + | | | |
| Streptococcus | + | + | | + | + | + | + | + | |
| constellatus | | | | | | | | | |
| Streptococcus criceti | + | + | | | | | | | |
| Streptococcus crista | + | + | | | | | | | |
| Streptococcus | + | + | | | | | | | |
| equisimilis | | | | | | | | | |
| Streptococcus | + | + | | | | | | | |
| gordonii | | | | | | | | | |
| Streptococcus | + | + | | | + | + | + | + | |
| intermedius | | | | | | | | | |
| Streptococcus mitis | + | + | + | | | | | | |
| Streptococcus mutans | + | + | | | | | | | |
| Streptococcus oralis | + | + | | | | | | | |
| Streptococcus | + | + | | | | | | | |
| parasanguis | | | | | | | | | |
| Streptococcus | + | | | | | | | | |
| pneumoniae | | | | | | | | | |
| Streptococcus | + | + | + | | | | | | + |
| pyogenes | | | | | | | | | |
| Streptococcus | + | + | + | | | | | | |
| salivarius | | | | | | | | | |
| Streptococcus | + | + | + | | | | | | |
| sanguis | | | | | | | | | |
| Streptococcus | + | + | | | | | | | |
| sobrinus | | | | | | | | | |
| Streptococcus | + | + | | | | | | | |
| vestibularis | | | | | | | | | |
| Group C + G | | + | | | | + | | | |
| Streptococci | | | | | | | | | |
| Succinivibrio | | | | + | + | + | | | |
| dextrinosolvens | | | | | | | | | |
| Sutterella spp. | + | + | | | + | + | + | | |
| Suttonella | + | + | | | | | | | |
| indologenes | | | | | | | | | |
| Tissierella praeacuta | | | | + | + | + | | | |
| Treponema denticola | + | + | | | | | | | |
| Treponema | + | + | | | | | | | |
| maltophilum | | | | | | | | | |
| Treponema minutum | | | | | | | + | | |
| Treponema | | | | | | | + | | |
| phagedenis | | | | | | | | | |
| Treponema | | | | | | | + | | |
| refringens | | | | | | | | | |
| Treponema | + | + | | | | | | | |
| socranskii | | | | | | | | | |
| Treponema vincentii | + | + | | | | | | | |
| Turicella otitidis | | | | | | | | | + |
| Ureaplasma | + | + | | | | | + | | |
| urealyticum | | | | | | | | | |
| Veillonella spp. | + | + | | + | + | + | | | |
| Weeksella virosa | | | | | | | + | + | |

Endogenous microbial flora, such as bacteria, have access to tissues for pathogenesis either through contiguous spread or bacteremic spread. Endogenous bacterial flora of the skin, mouth and colon are the species that are understood to be amenable to bacteremic spread, these and the other groups of endogenous organisms can spread by contiguous spread to adjacent tissues and organs. Bacteria that are members of a particular endogenous flora may therefore cause infection in tissues or organs to which the bacteria may spread. Accordingly, one aspect of the invention involves the use of microbial pathogens that are endogenous to a particular flora to treat a cancer of a tissue or organ to which the bacteria may spread to cause infection. The columns of Table 2 list 9 domains for endogenous flora, the: skin, respiratory system, genitals, GU system, mouth, stomach, duodenum/jejunum, ileum and colon. The rows of Table 2 list organs or tissues within which cancers may be situated. Accordingly, one aspect of the invention involves the use of microbial pathogens that are members of the endogenous flora to formulate antigenic compositions for treating cancers situated in tissues or organs to which the pathogen may spread to cause an infection. Accordingly, in alternative embodiments, tumors situated in the tissues or organs listed in the first column of Table 2 may be treated with antigenic compositions comprising antigenic determinants that are specific for microbial pathogens that are members of the endogenous flora of one or more of the tissue domains listed in the first row of Table 2, For example, tumors situated in the prostate may be treated with an antigenic composition having antigenic determinants specific for a microbial pathogen endogenous to the GU system. A number of the bacterial species that are endogenous to the tissue domains listed in Table 2 are listed, with the corresponding tissue domains, in Table 1. Accordingly, one aspect of the invention involves the treatment of a cancer situated in a tissue listed in Table 2 with an antigenic composition comprising antigenic determinants of the bacterial species that are listed in Table 1, where the regions of endogenous flora linked to the tumor in Table 2 match the regions of endogenous flora linked to the bacterial species in Table 1.

TABLE 2
Tissue/Organ Pathogenicity of Endogenous Flora

| Tissue/ | | | | GU | | | Duodenum/ | | |
|---|---|---|---|---|---|---|---|---|---|
| organ site | Skin | Respiratory | Genital | System | Mouth | Stomach | Jejunum | Ileum | Colon |
| Skin | X | | | | X | | | | |
| Soft tissue | X | | | | | | | | |
| (i.e. fat | | | | | | | | | |
| and | | | | | | | | | |
| muscle) | | | | | | | | | |
| (e.g., | | | | | | | | | |
| sarcoma) | | | | | | | | | |
| Breast | X | | | | X | | | | |
| Lymph | X | X | | | X | | | | |
| nodes: | | | | | | | | | |
| head and | | | | | | | | | |
| neck | | | | | | | | | |
| Lymph | X | | | | ✓ | | | | ✓ |
| nodes: | | | | | | | | | |
| axillae/ | | | | | | | | | |
| arm | | | | | | | | | |
| Lymph | | X | | | ✓ | | | | ✓ |
| nodes: | | | | | | | | | |
| mediastinal | | | | | | | | | |
| Lymph | | | | X | ✓ | X | X | X | X |
| nodes: | | | | | | | | | |
| intra- | | | | | | | | | |
| abdominal | | | | | | | | | |
| Lymph | X | | X | | ✓ | | | | ✓ |
| nodes: | | | | | | | | | |
| inguinal/ | | | | | | | | | |
| leg | | | | | | | | | |
| Hematological | ✓ | | | | ✓ | | | | ✓ |
| (e.g. | | | | | | | | | |
| leukemias, | | | | | | | | | |
| multiple | | | | | | | | | |
| myeloma, | | | | | | | | | |
| Bone | X | | | | ✓ | | | | ✓ |
| Meninges | | X | | | X | | | | |
| Brain | ✓ | | | | ✓ | | | | ✓ |
| Spinal | ✓ | | | | ✓ | | | | ✓ |
| cord | | | | | | | | | |
| Eye/Orbit | X | X | X | | X | | | | |
| Salivary | | | | | X | | | | |
| glands | | | | | | | | | |
| Oral | | | | | X | | | | |
| Tonsil | | X | | | X | | | | |
| Nasopharynx/ | | X | | | X | | | | |
| Sinus | | | | | | | | | |
| Thyroid | ✓ | | | | ✓ | | | | ✓ |
| Larynx | | X | | | X | | | | |
| Lung/Trac | | X | | | | | | | |
| Fhea/Bronchi | | | | | | | | | |
| Pleura | ✓ | X | | | ✓ | | | | ✓ |
| Mediastinum | | X | | | | | | | |
| Heart | ✓ | | | | ✓ | | | | ✓ |
| Esophagus | | | | | | X | | | |
| Stomach | | | | | | X | | | |
| Small | | | | | | | X | X | |
| bowel | | | | | | | | | |
| Colon/Rectum | | | | | | | | | X |
| Anus | X | | | | | | | | X |
| Perineum | X | | | | | | | | X |
| Liver | ✓ | | | | ✓ | | | | ✓ |
| Gallbladder | | | | | | | X | | |
| Billiary | | | | | | | X | | |
| tract | | | | | | | | | |
| Pancreas | | | | | | | X | | |
| Spleen | ✓ | | | | ✓ | | | | ✓ |
| Adrenal | ✓ | | | | ✓ | | | | ✓ |
| gland | | | | | | | | | |
| Kidney | ✓ | | | X | ✓ | | | | ✓ |
| Ureter | | | | X | | | | | |
| Bladder | ✓ | | X | X | | | | | |
| Peritoneum | | | | | | X | X | X | X |
| Retroperitoneal | | | | X | | X | X | X | X |
| area | | | | | | | | | |
| Prostate | | | X | X | | | | | |
| Testicle | | | X | X | | | | | |
| Penis | X | | X | X | | | | | |
| Ovary/Adnexae | | | X | X | | | | | |
| Uterus | | | X | X | | | | | X |
| Cervix | | | X | X | | | | | X |
| Vagina | | | X | | | | | | X |
| Vulva | | | X | | | | | | X |

*Bacteria have access to tissues/organs either through: Contiguous spread (X) or Bacteremic spread: (✓).

In accordance with the combined information in Tables 1 and 2, cancers located in the tissues or organs set out in column 1 of Table 2 may be treated with antigenic compositions comprising antigenic determinants of the corresponding bacteria of Table 1, so that the column headings in Table 2 are in effect replaced with the bacterial species of Table 1.

In some embodiments, microbial pathogens for use in the invention may be exongenous bacterial pathogens. For example, the organisms listed in Table 3 may be used as microbial pathogens to formulate antigenic compositions for use to treat cancers situated in the tissues or organs listed with the relevant organism in Table 3.

TABLE 3
Exogenous Bacterial Human Pathogens, and their Sites of Infection

| bacterial species | tissue/organ sites |
|---|---|
| Achromobacter | hematological, skin, soft tissue, lung/trachea/bronchi, |
| spp. | peritoneum, meninges, bile duct, gallbladder, kidney, |
| | bladder, ureter |
| Actinomadura | skin, soft tissue, lung/trachea/bronchi, mediastinum, brain, |
| spp. | spinal cord, hematological, meninges |
| Aerobacter spp. | small bowel, colon, hematological, peritoneum |
| Aerococcus spp. | hematological, heart, bone, kidney, bladder, ureter, meninges |
| Alcaligenes spp. | lung/trachea/bronchi |
| Anaplasma spp. | meninges, hematological, liver, spleen, bone, |
| | lung/trachea/bronchi |
| Bacillus anthracis | lung/trachea/bronchi, mediastinum, meninges, skin, |
| | nasopharynx, tonsil, oral, small bowel, colon, hematological |
| Bacillus cereus | colon, eye, hematological |
| other Bacillus | hematological, bone, meninges, brain, heart, |
| spp. | lung/trachea/bronchi, mediastinum, skin, soft tissue, colon, |
| | stomach, small bowel, eye |
| Balneatrix spp. | lung/trachea/bronchi, meninges, hematological |
| Bartonella | skin, hematological, liver, muscle, lymph nodes |
| bacilliformis | |
| Bartonella | brain, spinal cord, hematological, skin, liver, bone, pleura, |
| henselae | lung/trachea/bronchi, mediastinum, axillary and inguinal |
| | lymph nodes, eye |
| Bartonella quintana | skin, hematological, liver, spleen |
| Bergeyella | skin, soft tissue, meninges, hematological, |
| zoohelcum | lung/trachea/bronchi |
| Bordetella holmesii | lung/trachea/bronchi, hematological |
| Bordetella | nasopharynx, tonsil, lung/trachea/bronchi |
| parapertussis | |
| Bordetella pertussis | nasopharynx, tonsil, lung/trachea/bronchi |
| Borrelia | meninges, brain, spinal cord, skin, eye, hematological, |
| burgdorferi | inguinal/axillary/cervical lymph nodes, muscle, liver, |
| | spleen, nasopharynx, lung/trachea/bronchi, testes |
| Borrelia | brain, spinal cord, hematological, small bowel, liver, |
| recurrentis | spleen, salivary glands, lung/trachea/bronchi, lymph |
| | nodes, eye, skin |
| Brevundimonas | peritoneum, hematological, skin, soft tissue |
| spp. | |
| Brucella spp. | lung/trachea/bronchi, meninges, brain, spinal cord, lymph |
| | nodes, mediastinum, bone, eye, small bowel, colon, liver, |
| | biliary tract, kidney, ureter, bladder, hematological, skin, |
| | testes, spleen, prostate |
| Burkholderia gladioli | hematological, meninges, lung/trachea/bronchi |
| Burkholderia | lung/trachea/bronchi, skin, soft tissue, liver, spleen, |
| mallei | muscle, mediastinal lymph nodes, mediastinum, head and |
| | neck lymph nodes, hematological |
| Burkholderia | lung/trachea/bronchi, skin, kidney, bladder, ureter, soft |
| pseudomallei | tissue, bone, brain, spinal cord, muscle, hematological, |
| | prostate, kidney, ureter, meninges |
| Calymmatobacterium | skin, penis, vulva, soft tissue, vagina, cervix, bone, |
| granulomatis | hematological, inguinal lymph nodes |
| Campylobacter coli | small bowel, colon |
| Campylobacter | lung/trachea/bronchi, small bowel, colon, meninges, brain, |
| fetus | peritoneum, bone, gallbladder, ovaries, hematological, |
| | heart, kidney, bladder, ureter |
| Campylobacter | colon, hematological, gallbladder, pancreas, bladder, |
| jejuni | bone, meninges |
| Campylobacter sputorum | small bowel, colon |
| Capnoctyophaga | skin, soft tissue, meninges, hematological, bone, |
| canimorsus | lung/trachea/bronchi, eye |
| Capnoctyophaga | skin, soft tissue, meninges, hematological, bone, |
| cynodegmi | lung/trachea/bronchi, eye |
| CDC groups EF- | hematological, eye, skin, soft tissue |
| 4a and EF-4b | |
| Chlamydia | lung/trachea/bronchi, liver, brain, meninges, skin, thyroid, |
| pneumoniae | pancreas, hemantological |
| Chlamydia psittaci | lung/trachea/bronchi, mediastinum, liver, brain, meninges, |
| | hematological, skin, thyroid, pancreas |
| Chlamydia | inguinal lymph nodes, penis, vulva, vagina, cervix, uterus, |
| trachomatis | ovaries and adnexae, peritoneum, prostate, eye |
| Chlamydophila | laryngx, trachea/bronchi, hematological |
| pneumoniae | |
| Chromobacterium | hematological, liver, spleen, lung/trachea/bronchi, kidney, |
| violaceum | bladder, ureter, eye/orbit, bone, brain, meninges, spinal cord |
| Chlamydophila | lung/trachea/bronchi |
| psittaci | |
| Chryseobacterium | meninges, lung/trachea/bronchi, hematological |
| spp. | |
| Clostridium | small bowel, colon, stomach, skin, soft tissue, |
| bifermentans | hematological |
| Clostridium | colon, small bowel, skin |
| botulinum | |
| Clostridium | colon |
| difficile | |
| Clostridium indolis | small bowel, colon, stomach, skin, soft tissue, |
| | hematological |
| Clostridium | small bowel, colon, stomach, skin, soft tissue, |
| mangenolii | hematological |
| Clostridium | small bowel, colon, stomach, skin, soft tissue, |
| perfringens | hematological, heart |
| Clostridium | small bowel, colon, stomach, skin, soft tissue, |
| sordellii | hematological |
| Clostridium | small bowel, colon, stomach, skin, soft tissue, |
| sporogenes | hematological |
| Clostridium | small bowel, colon, stomach, skin, soft tissue, |
| subterminale | hematological |
| Clostridium tetani | skin, soft tissue |
| Comamonas spp. | hematological, peritoneum, eye |
| Corynebacterium | neckl/axillary/inguinal/mediastinal lymph nodes, |
| pseudotuberculosis | lung/trachea/bronchi, mediastinum |
| Coxiella burnetii | lung/bronchi/trachea, brain, spinal cord, liver, bone |
| Edwarsiella tarda | skin, soft tissue, liver, meninges, small bowel, colon, bone, |
| | uterus, ovaries |
| Ehrlichia spp. | meninges, brain, spinal cord, hematological, bone, liver, |
| | kidney, spleen, lymph nodes |
| Erysipelothrix | skin, hematological, bone, brain, peritoneum |
| rhusiopathiae | |
| Francisella | nasopharynx, oral, tonsil, lung/trachea/bronchi, skin, |
| tularensis | axillary/head and neck/inguinal lymph nodes, |
| | hematological, eye, small bowel |
| Fusobacterium | skin, soft tissue, hematological |
| spp. | |
| Gordonia spp. | skin, soft tissue, lung/trachea/bronchi, mediastinum, brain, |
| | spinal cord, hematological, meninges, eye |
| Haemophilus | skin, inguinal lymph nodes, penis, vulva, vagina |
| ducreyi | |
| Helicobacter | stomach |
| pylori | |
| Legionella spp. | lung/trachea/bronchi, hematological, brain, spinal cord, |
| | muscle, pancreas |
| Leptospirosis spp. | lung/trachea/bronchi, pancreas, meninges, brain, spinal |
| | cord, skin, lymph nodes, eye, hematological, nasopharynx, |
| | oral, tonsil, kidney, liver, spleen |
| Listeria | hematological, brain, meninges, spinal cord, small bowel, |
| monocytogenes | colon |
| Methylobacterium | hematological, peritoneum, skin, soft tissue, bone |
| spp. | |
| Mycobacterium | lung/bronchi/trachea, prostate, pancreas, spleen, skin, |
| avium | neck lymph nodes, esophagus, bone, hematological |
| Mycobacterium | colon, small bowel |
| bovis | |
| Mycobacterium | lung/bronchi/trachea, prostate, bone |
| kansasii | |
| Mycobacterium | skin, soft tissues, testes, eye |
| leprae | |
| Mycobacterium | skin, soft tissue, bone |
| marinum | |
| Mycobacterium | head and neck lymph nodes |
| scrofulaceum | |
| Mycobacterium | lung/bronchi/trachea, prostate, peritoneum, pancreas, |
| tuberculosis | spleen, lymph nodes, small bowel, meninges, brain, spinal |
| | cord, kidney, ureter, bladder, muscle, esophagus, colon, |
| | testes, eye, ovaries, cervix, vagina, uterus, mediastinum, |
| | larynx, skin, hematological, pleura |
| Mycobacterium | skin, soft tissue |
| ulcerans | |
| other | lung/bronchi/trachea, skin, soft tissues, bone, head and |
| Mycobacterium | neck lymph nodes |
| spp. | |
| Myroides spp. | kidney, bladder, ureter, skin, soft tissue, hematological |
| Neisseria | nasopharyx, oral, tonsil, prostate, penis, vagina, cervix, |
| gonorrhoeae | uterus, ovary/adnexae, peritoneum, skin, muscle, bone, |
| | liver, hematological, head and neck and inguinal and intra- |
| | abdominal lymph nodes, anus |
| Neorickettsia | hematological, bone, lymph nodes, liver, spleen |
| sennetsu | |
| Nocardia spp. | lung/bronchi/trachea, pancreas, meninges, spinal cord, |
| | brain, skin, soft tissue, eye, bone, kidney, heart, |
| | hematological |
| Orientia | meninges, brain, spinal cord, hematological, skin, inguinal |
| tsutsugamushi | and axillary lymph nodes, spleen, lung/bronchi/trachea |
| Pandoraea spp. | lung/trachea/bronchi, hematological |
| Pasteurella canis | skin, soft tissue, hematological |
| Pasteurella | skin, soft tissue, hematological |
| dagmatis | |
| Pasteurella | skin, soft tissue, hematological |
| stomatis | |
| Pediococcus spp. | hematological, liver, colon |
| Pityrosporum | skin |
| ovale | |
| Plesiomonas | small bowel, colon, hematological, meninges, bone, gall |
| shigelloides | bladder, skin, soft tissue |
| Pseudomonas | lung/trachea/bronchi, hemaotogical, skin, soft tissue, bone, |
| aeruginosa | meninges, brain, eye, kidney, bladder, ureter, heart |
| other | skin, soft tissue, lung/trachea/bronchi, mediastinum, |
| Pseudomonas | hematological |
| spp. | |
| Ralstonia spp. | hematological, meninges, bone |
| Rhizobium spp. | hematological, peritoneum, eye, kidney, bladder, ureter |
| Rhodococcus | lung/trachea/bronchi, hematological, brain, skin, lymph |
| spp. | nodes, bone, mediastinum, liver, spleen, soft tissue, spinal |
| | cord, meninges |
| Rickettsia akari | skin |
| Rickettsia conorii | lung/bronchi/trachea, meninges, brain, spinal cord, |
| | hematolofical, skin, kidney, liver, spleen, pancreas |
| Rickettsia felis | skin, brain, spinal cord |
| Rickettsia | meninges, brain, spincal cord, hematological, |
| prowazekii | lung/bronchi/trachea, skin, spleen |
| Rickettsia | lung/bronchi/trachea, meninges, brain, spinal cord, |
| rickettsiae | hematological, muscle, small bowel, liver, skin |
| Rickettsia slovaca | skin, head and neck lymph nodes |
| Rickettsia typhi | meninges, hematological, liver, kidney, brain, |
| | lung/bronchi/trachea, spleen |
| Roseomonas spp. | hematological, peritoneum, skin, soft tissue, bladder, |
| | kidney, ureter |
| Salmonella spp. | lung/bronchi/trachea, pancreas, spleen, intra-abdominal |
| | lymph nodes, stomach, small bowel, colon, meninges, |
| | skin, muscle, bone, hematological, heart |
| Shewanella spp. | skin, soft tissue, eye, bone, hematological, peritoneum |
| Shigella boydii | colon |
| Shigella | colon |
| dysenteriae | |
| Shigella flexneri | colon |
| Shigella sonnei | colon |
| Sphingobacterium | brain, meninges, spinal cord, eye, skin, soft tissue |
| spp. | |
| Sphingomonas | hematological, meninges, peritoneum, skin, soft tissue, |
| spp. | kidney, bladder, ureter |
| Spirillum minus | skin, axillary/inguinal/neck lymph nodes, hematological, |
| | liver, spleen |
| other Spirillum | colon |
| spp. | |
| Stenotrophomonas | meninges, hematological, peritoneum, |
| maltophilia | lung/trachea/bronchi, eye, kidney, bladder, ureter, skin, |
| | soft tissue |
| Streptobacillus | skin, bone, hematological, lung/trachea/bronchi, meninges, |
| moniliformis | brain, liver, spleen |
| Streptococcus | skin, hematological, soft tissue |
| iniae | |
| Streptococcus | small bowel, nasopharynx, bone, meninges, |
| zooepidemicus | hematological, head and neck lymph nodes |
| Streptomices spp. | skin, soft tissue, lung/trachea/bronchi, mediastinum, brain, |
| | spinal cord, hematological, meninges |
| Treponema | nasopharynx, tonsil, oral, meninges, brain, spinal cord, |
| pallidum | penis, vulva, vagina, anus, cervix, eye, hematological, |
| | inguinal and head and neck lymph nodes |
| Tropheryma | brain, spinal cord, hematological, small bowel, colon, |
| whipplei | heart, lung/trachea/bronchi, eye |
| Tsukamurella | skin, soft tissue, lung/trachea/bronchi, mediastinum, brain, |
| spp. | spinal cord, hematological, meninges |
| Vibrio cholerae | colon, small bowel |
| Vibrio | hematological, meninges |
| cincinnatiensis | |
| Vibrio damsela | skin, soft tissue |
| Vibrio fluvialis | small bowel, colon |
| Vibrio furnissii | small bowel, colon |
| Vibrio hollisae | small bowel, colon, skin, soft tissue |
| Vibrio | hematological |
| metschnikovii | |
| Vibrio | colon, small bowel |
| parahaemolyticus | |
| Vibrio vulnificus | soft tissue, blood, skin |
| Yersinia | nasopharynx, tonsil, small bowel, intra-abdominal lymph |
| enterocolitica | nodes, colon, muscle, lung/trachea/bronchi, liver, spleen, |
| | hematological |
| Yersinia pestis | lung/trachea/bronchi, inguinal/axillary/neck lymph nodes, |
| | oral, tonsil, hematological, skin |
| Yersinia | small bowel, colon, abdomincal lymph nodes |
| pseudotuberculosis | |

In some embodiments, microbial pathogens for use in the invention may be viral pathogens. Table 4 provides an exemplary list of viral pathogens together with the tissue and organ sites for which each viral species is reportedly a pathogen. Accordingly, one aspect of the invention involves utilizing immunogenic compositions that are specific for the named viruses to treat a cancer situated in the organs or tissues that are identified adjacent to the name of the virus in Table 3. For example, an antigenic composition derived from, or specific for, a vaccinia virus, may be used to treat a cancer situated in the skin, hematological tissues, lymph nodes, brain, spinal cord, eye or heart.

TABLE 4
Viral Human Pathogens and Their Sites of Infection

| virus | tissue/organ sites |
|---|---|
| Vaccinia | skin, hematological, lymph nodes, brain, spinal cord, eye, heart |
| Variola (smallpox) | skin, hematological, lymph nodes, brain |
| Monkeypox | skin, hematological, head and neck lymph nodes, brain, |
| | eye, lung/trachea/bronchi, mediastinum, nasopharynx |
| Cowpox | skin, hematological, lymph nodes |
| Parapoxviruses | skin |
| Molluscum | skin |
| contagiosum | |
| Tanapox | skin, hematological, axillary and inguinal lymph nodes |
| Herpes Simplex | nasopharynx, oral, tonsil, hematological, |
| virus (1 and 2) | lung/bronchi/trachea, pancreas, meninges, brain, spinal |
| | cord, inguinal and head/neck lymph nodes, penis, vulva, |
| | perineum, esophagus, liver, eye, skin, rectum, tonsil, |
| | mediastinum, anus, vagina, cervix |
| Varicella-zoster | nasopharynx, sinus, lung/trachea/bronchi, hematological, |
| | pancreas, meninges, brain, spinal cord, esophagus, liver, |
| | eye, skin, heart, mediastinum |
| Cytomegalovirus | nasopharynx, lymph nodes, tonsil, hematological, |
| | lung/trachea/bronchi, pancreas, abdomincal lymph |
| | nodes, brain, spinal cord, esophagus, small bowel, |
| | colon/recutm, eye, liver, heart, skin, mediastinum, |
| | esophagus |
| Epstein-Barr virus | nasopharynx, tonsil, oral, lymph nodes, hematological, |
| | lung, abdomincal lymph nodes, brain, spinal cord, |
| | muscles, esophagus, liver, heart, skin, spleen, kidney, |
| | muscle, heart, lung/trachea/bronchi, mediastinum |
| Human herpesvirus 6 | skin, hematological, lung/trachea/bronchi, brain, |
| | meninges, liver |
| Human herpesvirus 7 | skin, brain, liver |
| Human herpesvirus 8 | nasopharynx, tonsil, hematological, skin, spleen, head |
| | and neck lymph nodes |
| Simian herpes B virus | brain, spinal cord, skin, hematological, lymph nodes |
| Adenovirus | nasopharynx, oral, larynx, trachea, bronchi, lung, lymph |
| | nodes, meninges, brain, spinal cord, small bowel, colon, |
| | liver, intra-abdominal lymph nodes, mediastinum, |
| | bladder, sinus, hematological, ureter, kidney, bladder, |
| | thyroid, heart |
| BK virus | kidney |
| Human | skin, anus, penis, vulva, cervix, vagina, oral |
| papillomavirus | |
| Hepatitis B virus | liver, pancreas, hematological |
| Hepatitis D virus | liver |
| Parvovirus B19 | skin, hematological, nasopharynx, bone, kidney, heart, |
| | liver, brain, meninges |
| Orthoreoviruses | nasopharynx, small bowel, colon, oral, sinus, lymph |
| | nodes, skin, lung/trachea/bronchi, meninges, brain, |
| | spinal cord, liver |
| Orbiviruses | brain, muscle, hematological, |
| Coltiviruses | hematological, skin, muscle, oral, spleen, lymph nodes, |
| | meninges, brain |
| Rotaviruses | small bowel, colon, liver, hematological, pancreas, |
| | nasopharynx, billiary tract, meninges, brain |
| Alphaviruses | brain, spinal cord, small bowel, colon, hematological, |
| | skin, bone |
| Rubella | skin, hematological, head and neck lymph nodes, spleen, |
| | nasopharynx, bone, brain, tonsil, bronchi, liver, heart |
| Yellow fever virus | hematological, liver, lung/trachea/bronchi, kidney, |
| | adrenal gland, spleen, lymph nodes, stomach, kidney |
| Dengue fever virus | hematological, lymph nodes, skin, spleen, muscle, liver, |
| | brain, nasopharynx |
| Japanese | brain, hematological, spinal cord |
| encephalitis virus | |
| West Nile | brain, hematological, spinal cord, muscle, lymph nodes, |
| encephalitis virus | liver, spleen, pancreas, meninges |
| St. Louis | brain, hematological, spinal cord, meninges, muscle, |
| encephalitis virus | nasopharynx |
| Tick-borne | brain, hematological, spinal cord, muscle, meninges |
| encephalitis virus | |
| other Flaviviruses | hematological, brain, meninges, bone, muscles, skin, |
| | lymph nodes |
| Hepatitis C virus | hematological, liver |
| Hepatitis G virus | liver |
| Coronaviruses | nasopharynx, sinus, oral, tonsil, larynx, |
| | lung/trachea/bronchi, small bowel, colon, tonsil, |
| | hematological |
| Toroviruses | small bowel, colon, hematological |
| Parainfluenza | nasopharynx, sinus, tonsil, oral, larynx, |
| viruses | lung/trachea/bronchi, meninges, hematological, |
| | mediastinum |
| Mumps virus | salivary glands, pancreas, brain, spinal cord, liver, testes, |
| | hematological, meninges, ovaries, bone, heart, kidney, |
| | thyroid, prostate, breast |
| Respiratory syncytial | nasopharynx, tonsil, sinus, lung/trachea/bronchi, |
| virus | mediastinum, hematological, oral, pleura |
| Human | nasopharynx, lung/trachea/bronchi, tonsil, sinus, |
| metapneumovirus | mediastinum, hematological, oral, pleura, larynx, eye, |
| | skin, small bowel, colon |
| Rubeola | nasopharynx, sinus, hematological, lung/trachea/bronchi, |
| | intra-abdominal lymph nodes, meninges, brain, spinal |
| | cord, liver, spleen, lymph nodes, skin, thymus, eye, oral, heart |
| Hendra virus | brain, meninges, lung/trachea/bronchi, kidney, |
| | hematological, muscle, |
| Nipah virus | brain, meninges, spleen, lymph nodes, thymus, |
| | lung/trachea/bronchi, kidneys, brain, spinal cord, |
| | meninges, hematological |
| Vesicular stomatitis | hematological, muscle, oral, tonsil, nasopharyngeal, |
| virus | lymph nodes, small bowel, colon |
| Rabies virus | skin, meninges, brain, spinal cord, oral, nasopharynx, |
| | salivary glands, hematological |
| Lyssaviruses | brain, spinal cord |
| Influenza virus | nasopharynx, laryngx, lung/trachea/bronchi, meninges, |
| | muscle, hematological, mediastinum, muscle, sinus, |
| | tonsil, oral, eye, pleura, brain, spinal cord, salivary |
| | glands, thyroid, heart |
| California | hematological, brain, meninges |
| encephalitis virus | |
| Hantaviruses | hematological, kidney, eye, skin, oral, muscle, |
| | lung/trachea/bronchi |
| other Bunyaviruses | brain, hematological, muscle, meninges, spinal cord |
| Lymphocytic | hematological, muscle, lymph nodes, skin, brain, |
| choriomeningitis | meninges, testes, bone |
| virus | |
| Lassa virus | nasopharynx, brain, spinal cord, lung/trachea/bronchi, |
| | mediastinum, muscle, testes, eye, heart, |
| Machupo virus | brain, meninges, hematological, muscle, eye, skin, lymph |
| | nodes, nasopharynx, small bowel, colon |
| Junin virus | brain, meninges, hematological, muscle, eye, skin, lymph |
| | nodes, nasopharynx, small bowel, colon |
| Human T-Cell | hematological, skin, lymph nodes, muscle, eye, bone, |
| Lymphotropic | lung, spinal cord, brain |
| viruses | |
| Poliovirus | nasopharynx, lung/trachea/bronchi, small bowel, neck |
| | and intra-abdominal lymph nodes, colon, hematological, |
| | liver, spleen, skin, brain, spinal cord, meninges, heart |
| Coxsackieviruses | nasopharynx, larynx, oral, tonsil, lung/trachea/bronchi, |
| | mediastinum, pancreas, muscle, brain, meninges, small |
| | bowel, neck and intra-abdominal lymph nodes, colon, |
| | hematological, spleen, skin, eye, sinus, liver, testes, |
| | bone, pleura, salivary glands, heart |
| Echoviruses | nasopharynx, oral, tonsil, lung/trachea/bronchi, muscle, |
| | brain, meninges, small bowel, neck and intra-abdominal |
| | lymph nodes, colon, hematological, mediastinum, spleen, |
| | skin, eye, sinus, liver, pancreas, testes, bone, salivary |
| | glands, heart |
| other Enteroviruses | lung/trachea/bronchi, meninges, brain, skin, heart |
| Hepatitis A virus | small bowel, colon, hematological, liver, spleen, brain, |
| | spinal cord, gallbladder, pancreas, kidney |
| Rhinoviruses | nasopharynx, sinus, oral, tonsil, larynx, |
| | lung/trachea/bronchi |
| Noroviruses and | small bowel, colon |
| other Caliciviruses | |
| Astroviruses | small bowel, colon |
| Picobirnaviruses | small bowel, colon |
| Hepatitis E virus | liver, small bowel, colon, hematological |

The cumulative information in Tables 1 through 4 provides an extensive identification of microbial pathogens that may be used in the formulation of antigenic compositions of the invention, together with an identication of the tissues or organs in which these organisms are pathogenic, and accordingly the tissues or organs in which a cancer is situated that may be treated with the antigenic formulation. In some embodiments, the microbial pathogen selected for use in antigenic compositions of the invention may be one that is a relatively common cause of acute infection in the tissue or organ in which the cancer to be treated is situated. Table 5 identifies bacterial and viral pathogens of this kind, together with the tissues and organs in which they commonly cause infection. Accordingly, in selected embodiments, a cancer residing in a tissue identified in the first column o of Table 5 may be treated with an antigenic composition that comprises antigenic determinants for one or more of the pathogenic organisms listed in the second column of Table 5. For example, a cancer residing in the skin may be treated with an antigenic composition comprising antigenic determinants of one or more of the following organisms: Staphylococcus aureus, Beta hemolytic streptococci group A, B, C and G, Corynebacterium diptheriae, Corynebacterium ulcerans, Pseudomonas aeruginosa, rubeola, rubella, varicella-zoster, echoviruses, coxsackieviruses, adenovirus, vaccinia, herpes simplex, or parvo B19.

TABLE 5
Common Causes of Acute Infection (Bacterial and Viruses)
For Each Tissue/Organ Site

| Tissue/organ | Common Bacterial or Viral Pathogen of specific |
|---|---|
| site | tissue/organ site |
| Skin | Staphylococcus aureus, Beta hemolytic streptococci group |
| | A, B, C and G, Corynebacterium diptheriae, |
| | Corynebacterium ulcerans, Pseudomonas aeruginosa |
| | rubeola, rubella, varicella-zoster, echoviruses, |
| | coxsackieviruses, adenovirus, vaccinia, herpes simplex, |
| | parvo B19 |
| Soft tissue (i.e. | Streptococcus pyogenes, Staphylococcus aureus, |
| fat and muscle) | Clostridium perfringens, other Clostridium spp. |
| (e.g., sarcoma) | influenza, coxsackieviruses |
| Breast | Staphylococcus aureus, Streptococcus pyogenes |
| Lymph nodes: | Staphylococcus aureus, Streptococcus pyogenes |
| head and neck | Epstein-Barr, cytomegalovirus, adenovirus, measles, |
| | rubella, herpes simplex, coxsackieviruses, varicella-zoster |
| Lymph nodes: | Staphylococcus aureus, Streptococcus pyogenes |
| axillae/arm | measles, rubella, Epstein-Barr, cytomegalovirus, |
| | adenovirus, varicella-zoster |
| Lymph nodes: | viridans streptococci, Peptococcus spp., |
| mediastinal | PeptoStreptococcus spp., Bacteroides spp., Fusobacterium |
| | measles, rubella, Epstein-Barr, cytomegalovirus, varicella- |
| | zoster, adenovirus |
| Lymph nodes: | Yersinia enterocolitica, Yersinia pseudotuberculosis, |
| intra-abdominal | Salmonella spp., Streptococcus pyogenes, Escherichia coli, |
| | Staphylococcus aureus |
| | measles, rubella, Epstein-Barr, cytomegalovirus, varicella- |
| | zoster, adenovirus, influenza, coxsackieviruses |
| Lymph nodes: | Staphylococcus aureus, Streptococcus pyogenes |
| inguinal/leg | measles, rubella, Epstein-Barr, cytomegalovirus, herpes |
| | simplex |
| Hematological | Staphylococcus aureus, Streptococcus pyogenes, |
| (e.g. leukemias, | coagulase-negative staphylococci, Enterococcus spp., |
| multiple myeloma) | Escherichia coli, Klebsiella spp., Enterobacter spp., Proteus |
| | spp., Pseudomonas aeruginosa, Bacteroides fragilis, |
| | Streptococcus pneumoniae, group B streptococci |
| | rubeola, rubella, varicella-zoster, echoviruses, |
| | coxsackieviruses, adenovirus, Epstein-Barr, |
| | cytomegalovirus |
| Bone | Staphylococcus aureus, coagulase-negative staphylococci, |
| | Streptococcus pyogenes, Streptococcus pneumoniae, |
| | Streptococcus agalactiae, other streptococci spp., |
| | Escherichia coli, Pseudomonas spp., Enterobacter spp., |
| | Proteus spp., Serratia spp. |
| | parvovirus B19, rubella, hepatitis B |
| Meninges | Haemophilus influenzae, Neisseria meningitidis, |
| | Streptococcus pneumoniae, Streptococcus agalactiae, |
| | Listeria monocytogenes |
| | echoviruses, coxsackieviruses, other enteroviruses, mumps |
| Brain | Streptococcus spp. (including S. anginosus, S. constellatus, |
| | S. intermedius), Staphylococcus aureus, Bacteroides spp., |
| | Prevotella spp., Proteus spp., Escherichia coli, Klebsiella |
| | spp., Pseudomonas spp., Enterobacter spp., Borrelia burgdorferi |
| | coxsackieviruses, echoviruses, poliovirus, other |
| | enteroviruses, mumps, herpes simplex, varicella-zoster, |
| | flaviviruses, bunyaviruses |
| Spinal cord | Haemophilus influenzae, Neisseria meningitidis, |
| | Streptococcus pneumoniae, Streptococcus agalactiae, |
| | Listeria monocytogenes, Borrelia burgdorferi |
| | coxsackieviruses, echoviruses, poliovirus, other |
| | enteroviruses, mumps, herpes simplex, varicella-zoster, |
| | flaviviruses, bunyaviruses |
| Eye/Orbit | Staphylococcus aureus, Streptococcus pyogenes, |
| | Streptococcus pneumoniae, Streptococcus milleri, |
| | Escherichia coli, Bacillus cereus, Chlamydia trachomatis, |
| | Haemophilus influenza, Pseudomonas spp., Klebsiella |
| | spp., Treponema pallidum |
| | adenoviruses, herpes simplex, varicella-zoster, |
| | cytomegalovirus |
| Salivary glands | Staphylococcus aureus, viridans streptococci (e.g., |
| | Streptococcus salivarius, Streptococcus sanguis, |
| | Streptococcus mutans), PeptoStreptococcus spp., |
| | Bacteroides spp., and other oral anaerobes |
| | mumps, influenza, enteroviruses, rabies |
| Oral | Prevotella melaninogenicus, anaerobic streptococci, |
| | viridans streptococci, Actinomyces spp., |
| | PeptoStreptococcus spp., Bacteroides spp., and other oral |
| | anaerobes |
| | herpes simplex, coxsackieviruses, Epstein-Barr |
| Tonsil | Streptococcus pyogenes, Group C and G B-hemolytic |
| | streptococci |
| | rhinoviruses, influenza, coronavirus, adenovirus, |
| | parainfluenza, respiratory syncytial virus, herpes simplex |
| Sinus | Streptococcus pneumoniae, Haemophilus influenza, |
| | Moraxella catarrhalis, α-streptococci, anaerobic bacteria |
| | (e.g., Prevotella), Staphylococcus aureus |
| | rhinoviruses, influenza, adenovirus, parainfluenza |
| Nasopharynx | Streptococcus pyogenes, Group C and G B-hemolytic |
| | streptococci |
| | rhinoviruses, influenza, coronavirus, adenovirus, |
| | parainfluenza, respiratory syncytial virus, herpes simplex |
| Thyroid | Staphylococcus aureus, Streptococcus pyogenes, |
| | Streptococcus pneumoniae |
| | mumps, influenza |
| Larynx | Mycoplasma pneumoniae, Chlamydophila pneumoniae, |
| | Streptococcus pyogenes |
| | rhinovirus, influenza, parainfluenza, adenovirus, corona |
| | virus, human metapneumovirus |
| Trachea | Mycoplasma pneumoniae |
| | parainfluenza, influenza, respiratory syncytial virus, |
| | adenovirus |
| Bronchi | Mycoplasma pneumoniae, Chlamydophila pneumoniae, |
| | Bordetella pertussis, Streptococcus pneumoniae, |
| | Haemophilus influenzae |
| | influenza, adenovirus, rhinovirus, coronavirus, |
| | parainfluenza, respiratory syncytial virus, human |
| | metapneumovirus, coxsackievirus |
| Lung | Streptococcus pneumoniae, Moraxella catarrhalis, |
| | Mycoplasma pneumoniae, Klebsiella pneumoniae, |
| | Haemophilus influenza, Staphylococcus aureus |
| | influenza, adenovirus, respiratory syncytial virus, |
| | parainfluenza |
| Pleura | Staphylococcus aureus, Streptococcus pyogenes, |
| | Streptococcus pneumoniae, Haemophilus influenzae, |
| | Bacteroides fragilis, Prevotella spp., Fusobacterium |
| | nucleatum, peptostreptococcus, Mycobacterium |
| | tuberculosis |
| | influenza, adenovirus, respiratory syncytial virus, |
| | parainfluenza |
| Mediastinum | viridans streptococci, Peptococcus spp., |
| | PeptoStreptococcus spp., Bacteroides spp., Fusobacterium spp. |
| | measles, rubella, Epstein-Barr, cytomegalovirus |
| Heart | Streptococcus spp. (including S. mitior, S. bovis, S. sanguis, |
| | S. mutans, S. anginosus), Enterococcus spp., |
| | Staphylococcus spp., Corynebacterium diptheriae, |
| | Clostridium perfringens, Neisseria meningitidis, |
| | Salmonella spp. |
| | enteroviruses, coxsackieviruses, echoviruses, poliovirus, |
| | adenovirus, mumps, rubeola, influenza |
| Esophagus | Actinomyces spp., Mycobacterium avium, Mycobacterium |
| | tuberculosis, Streptococcus spp. |
| | cytomegalovirus, herpes simplex, varicella-zoster |
| Stomach | Streptococcus pyogenes |
| | cytomegalovirus, herpes simplex, Epstein-Barr, rotaviruses, |
| | noroviruses, adenoviruses |
| Small bowel | Escherichia coli, Clostridium difficile, Bacteroides fragilis, |
| | Bacteroides vulgatus, Bacteroides thetaiotaomicron, |
| | Clostridium perfringens, Salmonella enteriditis, Yersinia |
| | enterocolitica, Shigella flexneri |
| | adenoviruses, astroviruses, caliciviruses, noroviruses, |
| | rotaviruses, cytomegalovirus |
| Colon/Rectum | Escherichia coli, Clostridium difficile, Bacteroides fragilis, |
| | Bacteroides vulgatus, Bacteroides thetaiotaomicron, |
| | Clostridium perfringens, Salmonella enteriditis, Yersinia |
| | enterocolitica, Shigella flexneri |
| | adenoviruses, astroviruses, caliciviruses, noroviruses, |
| | rotaviruses, cytomegalovirus |
| Anus | Streptococcus pyogenes, Bacteroides spp., Fusobacterium |
| | spp., anaerobic streptococci, Clostridium spp., E. coli, |
| | Enterobacter spp., Pseudomonas aeruginosa, Treponema |
| | pallidum |
| | herpes simplex |
| Perineum | Escherichia coli, Klebsiella spp., Enterococcus spp., |
| | Bacteroides spp., Fusobacterium spp., Clostridium spp., |
| | Pseudomonas aeruginosa, anaerobic streptococci, |
| | Clostridium spp., E. coli, Enterobacter spp., |
| | herpes simplex |
| Liver | Escherichia coli, Klebsiella spp., Streptococcus (anginosus |
| | group), Enterococcus, spp. other viridans streptococci, |
| | Bacteroides spp. |
| | hepatitis A, Epstein-Barr, herpes simplex, mumps, rubella, |
| | rubeola, varicella-zoster, coxsackieviruses, adenovirus |
| Gallbladder | Escherichia coli, Klebsiella spp., Enterobacter spp., |
| | enterococci, Bacteroides spp., Fusobacterium spp., |
| | Clostridium spp., Salmonella enteriditis, Yersinia |
| | enterocolitica, Shigella flexneri |
| Biliary tract | Escherichia coli, Klebsiella spp., Enterobacter spp., |
| | enterococci, Bacteroides spp., Fusobacterium spp., |
| | Clostridium spp., Salmonella enteriditis, Yersinia |
| | enterocolitica, Shigella flexneri |
| | hepatitis A, Epstein-Barr, herpes simplex, mumps, rubella, |
| | rubeola, varicella-zoster, cocsackieviruses, adenovirus |
| Pancreas | Escherichia coli, Klebsiella spp., Enterococcus spp., |
| | Pseudomonas spp., Staphylococcal spp., Mycoplasma, |
| | Salmonella typhi, Leptospirosis spp., Legionella |
| | mumps, coxsackievirus, hepatitis B, cytomegalovirus, |
| | herpes simplex 2, varicella-zoster |
| Spleen | Streptococcus spp., Staphylococcus spp., Salmonella spp., |
| | Pseudomonas spp., Escherichia coli, Enterococcus spp. |
| | Epstein-Barr, cytomegalovirus, adenovirus, measles, |
| | rubella, coxsackieviruses, varicella-zoster |
| Adrenal gland | Streptococcus spp., Staphylococcus spp., Salmonella spp., |
| | Pseudomonas spp., Escherichia coli, Enterococcus spp. |
| | varicella-zoster |
| Kidney | Escherichia coli, Proteus mirabilis, Proteus vulgatus, |
| | Providentia spp., Morganella spp., Enterococcus faecalis, |
| | Pseudomonas aeruginosa |
| | BK virus, mumps |
| Ureter | Escherichia coli, Proteus mirabilis, Proteus vulgatus, |
| | Providentia spp., Morganella spp., Enterococcus spp. |
| Bladder | Escherichia coli, Proteus mirabilis, Proteus vulgatus, |
| | Providentia spp., Morganella spp., Enterococcus faecalis, |
| | Corynebacterium jekeum |
| | adenovirus, cytomegalovirus |
| Peritoneum | Staphylococcus aureus, Streptococcus pyogenes, |
| | Streptococcus pneumonia, Escherichia coli, Klebsiella spp., |
| | Proteus spp., enterococci, Bacteroides fragilis, Prevotella |
| | melaninogenica, Peptococcus spp., Peptostreptococcus |
| | spp., Fusobacterium, Clostridium spp. |
| Retroperitoneal | Escherichia coli, Staphylococcus aureus |
| area | |
| Prostate | Escherichia coli, Klebsiella spp., Enterobacter spp., Proteus |
| | mirabilis, enterococci, Pseudomonas spp., |
| | Corynebacterium spp., Neisseria gonorrhoeae |
| | herpes simplex |
| Testicle | Escherichia coli, Klebsiella pneumoniae, Pseudomonas |
| | aeruginosa, Staphylococcus spp., Streptococcus spp., |
| | Salmonella enteriditis |
| | mumps, coxsackievirus, lymphocytic choriomeningitis virus |
| Penis | Staphylococcus aureus, Streptococcus pyogenes, |
| | Neisseria gonorrhoeae, Treponema pallidum |
| | herpes simplex, human papillomavirus |
| Ovary/Adnexae | Neisseria gonorrhoeae, Chlamydia trachomatis, |
| | Gardenerella vaginalis, Prevotella spp., Bacteroides spp., |
| | Peptococcus spp. Streptococcus spp., Escherichia coli |
| Uterus | Neisseria gonorrhoeae, Chlamydia trachomatis, |
| | Gardenerella vaginalis, Prevotella spp., Bacteroides spp., |
| | Peptococcus spp., Streptococcus spp., Escherichia coli |
| Cervix | Neisseria gonorrhoeae, Chlamydia trachomatis, |
| | Treponema pallidum |
| | herpes simplex |
| Vagina | Gardenerella vaginalis, Prevotella spp., Bacteroides spp., |
| | peptococci spp., Escherichia coli, Neisseria gonorrhoeae, |
| | Chlamydia trachomatis, Treponema pallidum, |
| | herpes simplex |
| Vulva | Staphylococcus aureus, Streptococcus pyogenes, |
| | Treponema pallidum |
| | herpes simplex |

In selected embodiments, particular microbial pathogens may be suited for treatment of particular cancers, examples of selected embodiments are set out in the Table 5. These are exemplary embodiments, and not an exhaustive list of the alternative formulations for use in accordance with the invention.

In some embodiments, selected compositions and methods are specifically excluded from the scope of the invention. For example, the use of the following microbial pathogens in the treatment of the following cancers is excluded from some embodiments, so that the claimed invention may extend to particular embodiments with the exception of one or more of the following:
BCG (Mycobacterium bovis) for the treatment of stomach cancer and colon cancer;
Mycobacterium w for the treatment of lung cancer;
Mycobacterium vaccae for the treatment of non-small-cell lung cancer;
Corynebacterium parvum for the treatment of melanoma;
Streptococcus pyogenes for the treatment of stomach cancer
Nocardia rubra for the treatment of lung cancer or acute myelogenous leukemia;
Lactobacillus casei for the treatment of cervical cancer;
Pseudomonas aeruginosa for the treatment of lymphoma and lung cancer;
Vaccinia for the treatment of melanoma; and
Rabies virus for the treatment of melanoma.

### Other Embodiments

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications are incorporated herein by reference as if each individual publication were specifically and individually indicated to be incorporated by reference herein and as though fully set forth herein. The invention includes all embodiments and variations substantially as hereinbefore described and with reference to the examples and drawings.

What is claimed is:

1. A method for formulating an immunogenic composition for treating a cancer situated in a specific organ or tissue in a mammal, comprising:
selecting at least one microbial pathogen that is pathogenic in the specific organ or tissue of the mammal within which the cancer is situated;
producing an antigenic composition comprising antigenic determinants that together are specific for the microbial pathogen; and,
formulating the antigenic composition for subcutaneous or intradermal injection, to produce the immunogenic composition.

2. The method of claim 1, wherein the mammal is human.

3. The method of claim 1, further comprising a diagnostic step of identifying the specific organ or tissue within which the cancer is situated prior to producing the antigenic composition.

4. The method of claim 1, wherein the antigenic composition is formulated for producing a localized skin inflammation at a site of administration.

5. The method of claim 1, wherein the organ or tissue is selected from the group consisting of prostate, lung, colon, rectum, bladder, oral, skin, stomach, pancreas, brain, larynx, esophagus, hematological, breast, uterus, ovary, thyroid, cervix, small bowel, anus, peritoneum, liver, spleen, kidney, adrenal gland, vagina, meninges, spinal cord, orbit, salivary glands, tongue, naso-pharyngeal, tonsil, bone, bone marrow, muscle, soft tissue, heart, mediastinum, retroperitoneal area, head and neck lymph nodes, axillae lymph nodes, chest lymph nodes, abdominal lymph nodes, inguinal lymph nodes.

6. The method of claim 1, wherein the cancer is a metastatic cancer having a site of metastasis, and the organ or tissue is a site of metastsis.

7. The method of claim 1, wherein the antigenic composition is formulated for repeated subcutaneous or intradermal administration.

8. The method of claim 1, wherein the microbial pathogen is a bacteria.

9. The method of claim 8, further comprising killing the bacteria to formulate the antigenic composition.

10. The method of claim 8, wherein the mammal has an endogenous flora and the bacteria is a member of the endogenous flora.

11. The method of claim 10, wherein the bacteria is endogenous to flora of the respiratory system, mouth, stomach, duodenum, jejunum, ileum, colon, genitourinary (GU) system, vagina or skin.

12. The method of claim 1, wherein the antigenic composition is capable of eliciting an adaptive immune response in the mammal specific to the microbial pathogen.

13. The method of claim 1, wherein the microbial pathogen is a virus.

14. The method of claim 10, wherein the organ or tissue is x, and the bacteria is endogenous to flora of the y of the mammal, and:
when x is prostate, y is GU system;
when x is lung, y is respiratory system;
when x is colon, y is rectum or colon;
when x is bladder, y is genitals;
when x is oral, y is mouth;
when x is skin, y is skin;
when x is stomach, y is stomach;
when x is pancreas, y is duodenum or jejunum;
when x is brain, y is skin or mouth or colon;
when x is larynx, y is respiratory tissue;
when x is esophagus, y is stomach;
when x is hematological, y is skin or mouth or colon;
when x is leukemia, y is skin or mouth or colon;
when x is multiple myeloma, y is skin or mouth or colon;
when x is breast, y is skin or mouth;
when x is uterus, y is genitals or GU system or colon;
when x is ovary, y is genitals or GU system or colon;
when x is thyroid, y is skin mouth or colon;
when x is cervix, y is genitals or GU system or colon;
when x is small bowel, y is duodenum or jejunum or ileum;
when x is anus, y is skin or colon;
when x is peritoneum, y is stomach or duodenum or jejunum or ileum or colon;
when x is kidney, y is GU system;
when x is vagina, y is genitals;
when x is salivary gland, y is mouth;
when x is tongue, y is mouth;
when x is nasopharyngeal, y is respiratory system or mouth;
when x is tonsil, y is respiratory system or mouth;
when x is muscle, y is skin;
when x is sarcoma, y is skin;
when x is head and neck lymph nodes, y is skin or respiratory or mouth;
when x is axillae lymph nodes, y is skin;
when x is chest lymph nodes, y is respiratory system;
when x is abdominal lymph nodes, y is GU system or duodenum or jejunum or ileum or colon;
when x is inguinal lymph nodes, y is skin or genitals.

15. The method of claim 1, further comprising formulating the antigenic composition with an NSAID.

16. A method of treating a mammal for a cancer situated in a tissue or an organ, the method comprising administering to the subject an effective amount of an antigenic composition comprising antigenic determinants that together are specific for at least one microbial pathogen, the microbial pathogen being pathogenic in the specific organ or tissue of the mammal within which the cancer is situated, and wherein the antigenic composition is administered at an administration site in successive doses given at a dosage interval of between one hour and one month, over a dosage duration of at least two weeks, wherein each dose is effective to cause visible localized inflammation at the administration site.

17. The method of claim 16, wherein the mammal is a human.

18. The method of claim 16, wherein the antigenic composition is administered intradermally or subcutaneously.

19. The method of claim 16, wherein the visible localized inflammation at the administration site occurs within 1 to 48 hours.

20. The method of claim 16, wherein there are two or more alternative sites of administration.

21. The method of claim 16, wherein each successive does is administered at a site of administration that is different from the site of administration of the previous dose.

22. The method of claim 16, wherein the dosage interval is 6 to 60 hours.

23. The method of claim 16, wherein the dosage duration is at least 2 weeks.

24. The method of claim 16, wherein the dosage duration is at least 2 months.

25. The method of claim 16, wherein further comprising treating the mammal with an effective amount of a NSAID.

26. The method of claim 16, wherein the tissue or organ is X, and the microbial pathogen is Y, wherein:

| when X is: | Y is one or more of: |
|---|---|
| Skin | Staphylococcus aureus, Beta hemolytic streptococci |
| | group A, B, C and G, Corynebacterium diptheriae, |
| | Corynebacterium ulcerans, Pseudomonas aeruginosa |
| | rubeola, rubella, varicella-zoster, echoviruses, |
| | coxsackieviruses, adenovirus, vaccinia, herpes simplex, parvo B19; |
| Soft tissue (i.e. | Streptococcus pyogenes, Staphylococcus aureus, |
| fat and muscle) | Clostridium perfringens, other Clostridium spp. |
| (e.g., sarcoma) | influenza, coxsackieviruses; |
| Breast | Staphylococcus aureus, Streptococcus pyogenes; |
| Lymph nodes: | Staphylococcus aureus, Streptococcus pyogenes |
| head and neck | Epstein-Barr, cytomegalovirus, adenovirus, measles, |
| | rubella, herpes simplex, coxsackieviruses, varicella-zoster; |
| Lymph nodes: | Staphylococcus aureus, Streptococcus pyogenes |
| axillae/arm | measles, rubella, Epstein-Barr, cytomegalovirus, |
| | adenovirus, varicella-zoster; |
| Lymph nodes: | viridans streptococci, Peptococcus spp., |
| mediastinal | PeptoStreptococcus spp., Bacteroides spp., Fusobacterium |
| | measles, rubella, Epstein-Barr, cytomegalovirus, |
| | varicella-zoster, adenovirus; |
| Lymph nodes: | Yersinia enterocolitica, Yersinia pseudotuberculosis, |
| intra-abdominal | Salmonella spp., Streptococcus pyogenes, |
| | Escherichia coli, Staphylococcus aureus |
| | measles, rubella, Epstein-Barr, cytomegalovirus, |
| | varicella-zoster, adenovirus, influenza, coxsackieviruses; |
| Lymph nodes: | Staphylococcus aureus, Streptococcus pyogenes |
| inguinal/leg | measles, rubella, Epstein-Barr, cytomegalovirus, herpes simplex; |
| Hematological | Staphylococcus aureus, Streptococcus pyogenes, |
| (e.g. leukemias, | coagulase-negative staphylococci, Enterococcus spp., |
| multiple myeloma) | Escherichia coli, Klebsiella spp., Enterobacter spp., |
| | Proteus spp., Pseudomonas aeruginosa, Bacteroides |
| | fragilis, Streptococcus pneumoniae, group B streptococci |
| | rubeola, rubella, varicella-zoster, echoviruses, |
| | coxsackieviruses, adenovirus, Epstein-Barr, cytomegalovirus; |
| Bone | Staphylococcus aureus, coagulase-negative |
| | staphylococci, Streptococcus pyogenes, Streptococcus |
| | pneumoniae, Streptococcus agalactiae, other streptococci |
| | spp., Escherichia coli, Pseudomonas spp., Enterobacter |
| | spp., Proteus spp., Serratia spp. |
| | parvovirus B19, rubella, hepatitis B; |
| Meninges | Haemophilus influenzae, Neisseria meningitidis, |
| | Streptococcus pneumoniae, Streptococcus agalactiae, |
| | Listeria monocytogenes |
| | echoviruses, coxsackieviruses, other enteroviruses, mumps; |
| Brain | Streptococcus spp. (including S. anginosus, S. constellatus, |
| | S. intermedius), Staphylococcus aureus, |
| | Bacteroides spp., Prevotella spp., Proteus spp., |
| | Escherichia coli, Klebsiella spp., Pseudomonas spp., |
| | Enterobacter spp., Borrelia burgdorferi |
| | coxsackieviruses, echoviruses, poliovirus, other |
| | enteroviruses, mumps, herpes simplex, varicella-zoster, |
| | flaviviruses, bunyaviruses; |
| Spinal cord | Haemophilus influenzae, Neisseria meningitidis, |
| | Streptococcus pneumoniae, Streptococcus agalactiae, |
| | Listeria monocytogenes, Borrelia burgdorferi |
| | coxsackieviruses, echoviruses, poliovirus, other |
| | enteroviruses, mumps, herpes simplex, varicella-zoster, |
| | flaviviruses, bunyaviruses; |
| Eye/Orbit | Staphylococcus aureus, Streptococcus pyogenes, |
| | Streptococcus pneumoniae, Streptococcus milleri, |
| | Escherichia coli, Bacillus cereus, Chlamydia trachomatis, |
| | Haemophilus influenza, Pseudomonas spp., Klebsiella |
| | spp., Treponema pallidum |
| | adenoviruses, herpes simplex, varicella-zoster, |
| | cytomegalovirus; |
| Salivary glands | Staphylococcus aureus, viridans streptococci (e.g., |
| | Streptococcus salivarius, Streptococcus sanguis, |
| | Streptococcus mutans), PeptoStreptococcus spp., |
| | Bacteroides spp., and other oral anaerobes |
| | mumps, influenza, enteroviruses, rabies; |
| Oral | Prevotella melaninogenicus, anaerobic streptococci, |
| | viridans streptococci, Actinomyces spp., |
| | PeptoStreptococcus spp., Bacteroides spp., and other oral |
| | anaerobes |
| | herpes simplex, coxsackieviruses, Epstein-Barr; |
| Tonsil | Streptococcus pyogenes, Group C and G B-hemolytic |
| | streptococci |
| | rhinoviruses, influenza, coronavirus, adenovirus, |
| | parainfluenza, respiratory syncytial virus, herpes simplex; |
| Sinus | Streptococcus pneumoniae, Haemophilus influenza, |
| | Moraxella catarrhalis, α-streptococci, anaerobic bacteria |
| | (e.g., Prevotella), Staphylococcus aureus |
| | rhinoviruses, influenza, adenovirus, parainfluenza; |
| Nasopharynx | Streptococcus pyogenes, Group C and G B-hemolytic |
| | streptococci |
| | rhinoviruses, influenza, coronavirus, adenovirus, |
| | parainfluenza, respiratory syncytial virus, herpes simplex; |
| Thyroid | Staphylococcus aureus, Streptococcus pyogenes, |
| | Streptococcus pneumoniae |
| | mumps, influenza; |
| Larynx | Mycoplasma pneumoniae, Chlamydophila pneumoniae, |
| | Streptococcus pyogenes |
| | rhinovirus, influenza, parainfluenza, adenovirus, coronavirus |
| | virus, human metapneumovirus; |
| Trachea | Mycoplasma pneumoniae |
| | parainfluenza, influenza, respiratory syncytial virus, |
| | adenovirus; |
| Bronchi | Mycoplasma pneumoniae, Chlamydophila pneumoniae, |
| | Bordetella pertussis, Streptococcus pneumoniae, |
| | Haemophilus influenzae |
| | influenza, adenovirus, rhinovirus, coronavirus, |
| | parainfluenza, respiratory syncytial virus, human |
| | metapneumovirus, coxsackievirus; |
| Lung | Streptococcus pneumoniae, Moraxella catarrhalis, |
| | Mycoplasma pneumoniae, Klebsiella pneumoniae, |
| | Haemophilus influenza, Staphylococcus aureus |
| | influenza, adenovirus, respiratory syncytial virus, |
| | parainfluenza; |
| Pleura | Staphylococcus aureus, Streptococcus pyogenes, |
| | Streptococcus pneumoniae, Haemophilus influenzae, |
| | Bacteroides fragilis, Prevotella spp., Fusobacterium |
| | nucleatum, peptostreptococcus, Mycobacterium |
| | tuberculosis |
| | influenza, adenovirus, respiratory syncytial virus, |
| | parainfluenza; |
| Mediastinum | viridans streptococci, Peptococcus spp., |
| | PeptoStreptococcus spp., Bacteroides spp., |
| | Fusobacterium spp. |
| | measles, rubella, Epstein-Barr, cytomegalovirus; |
| Heart | Streptococcus spp. (including S. mitior, S. bovis, S. sanguis, |
| | S. mutans, S. anginosus), Enterococcus spp., |
| | Staphylococcus spp., Corynebacterium diptheriae, |
| | Clostridium perfringens, Neisseria meningitidis, |
| | Salmonella spp. |
| | enteroviruses, coxsackieviruses, echoviruses, poliovirus, |
| | adenovirus, mumps, rubeola, influenza; |
| Esophagus | Actinomyces spp., Mycobacterium avium, Mycobacterium |
| | tuberculosis, Streptococcus spp. |
| | cytomegalovirus, herpes simplex, varicella-zoster; |
| Stomach | Streptococcus pyogenes |
| | cytomegalovirus, herpes simplex, Epstein-Barr, |
| | rotaviruses, noroviruses, adenoviruses; |
| Small bowel | Escherichia coli, Clostridium difficile, Bacteroides fragilis, |
| | Bacteroides vulgatus, Bacteroides thetaiotaomicron, |
| | Clostridium perfringens, Salmonella enteriditis, Yersinia |
| | enterocolitica, Shigella flexneri |
| | adenoviruses, astroviruses, caliciviruses, noroviruses, |
| | rotaviruses, cytomegalovirus; |
| Colon/Rectum | Escherichia coli, Clostridium difficile, Bacteroides fragilis, |
| | Bacteroides vulgatus, Bacteroides thetaiotaomicron, |
| | Clostridium perfringens, Salmonella enteriditis, Yersinia |
| | enterocolitica, Shigella flexneri |
| | adenoviruses, astroviruses, caliciviruses, noroviruses, |
| | rotaviruses, cytomegalovirus; |
| Anus | Streptococcus pyogenes, Bacteroides spp., |
| | Fusobacterium spp., anaerobic streptococci, Clostridium |
| | spp., E. coli, Enterobacter spp., Pseudomonas |
| | aeruginosa, Treponema pallidum |
| | herpes simplex; |
| Perineum | Escherichia coli, Klebsiella spp., Enterococcus spp., |
| | Bacteroides spp., Fusobacterium spp., Clostridium spp., |
| | Pseudomonas aeruginosa, anaerobic streptococci, |
| | Clostridium spp., E. coli, Enterobacter spp., |
| | herpes simplex; |
| Liver | Escherichia coli, Klebsiella spp., Streptococcus |
| | (anginosus group), Enterococcus, spp. other viridans |
| | streptococci, Bacteroides spp. |
| | hepatitis A, Epstein-Barr, herpes simplex, mumps, |
| | rubella, rubeola, varicella-zoster, coxsackieviruses, |
| | adenovirus; |
| Gallbladder | Escherichia coli, Klebsiella spp., Enterobacter spp., |
| | enterococci, Bacteroides spp., Fusobacterium spp., |
| | Clostridium spp., Salmonella enteriditis, Yersinia |
| | enterocolitica, Shigella flexneri; |
| Biliary tract | Escherichia coli, Klebsiella spp., Enterobacter spp., |
| | enterococci, Bacteroides spp., Fusobacterium spp., |
| | Clostridium spp., Salmonella enteriditis, Yersinia |
| | enterocolitica, Shigella flexneri |
| | hepatitis A, Epstein-Barr, herpes simplex, mumps, |
| | rubella, rubeola, varicella-zoster, cocsackieviruses, |
| | adenovirus; |
| Pancreas | Escherichia coli, Klebsiella spp., Enterococcus spp., |
| | Pseudomonas spp., Staphylococcal spp., Mycoplasma, |
| | Salmonella typhi, Leptospirosis spp., Legionella |
| | mumps, coxsackievirus, hepatitis B, cytomegalovirus, |
| | herpes simplex 2, varicella-zoster; |
| Spleen | Streptococcus spp., Staphylococcus spp., Salmonella |
| | spp., Pseudomonas spp., Escherichia coli, Enterococcus spp. |
| | Epstein-Barr, cytomegalovirus, adenovirus, measles, |
| | rubella, coxsackieviruses, varicella-zoster; |
| Adrenal gland | Streptococcus spp., Staphylococcus spp., Salmonella |
| | spp., Pseudomonas spp., Escherichia coli, Enterococcus spp. |
| | varicella-zoster; |
| Kidney | Escherichia coli, Proteus mirabilis, Proteus vulgatus, |
| | Providentia spp., Morganella spp., Enterococcus faecalis, |
| | Pseudomonas aeruginosa |
| | BK virus, mumps; |
| Ureter | Escherichia coli, Proteus mirabilis, Proteus vulgatus, |
| | Providentia spp., Morganella spp., Enterococcus spp.; |
| Bladder | Escherichia coli, Proteus mirabilis, Proteus vulgatus, |
| | Providentia spp., Morganella spp., Enterococcus faecalis, |
| | Corynebacterium jekeum |
| | adenovirus, cytomegalovirus; |
| Peritoneum | Staphylococcus aureus, Streptococcus pyogenes, |
| | Streptococcus pneumonia, Escherichia coli, Klebsiella |
| | spp., Proteus spp., enterococci, Bacteroides fragilis, |
| | Prevotella melaninogenica, Peptococcus spp., |
| | PeptoStreptococcus spp., Fusobacterium, Clostridium spp.; |
| Retroperitoneal | Escherichia coli, Staphylococcus aureus; |
| area | |
| Prostate | Escherichia coli, Klebsiella spp., Enterobacter spp., |
| | Proteus mirabilis, enterococci, Pseudomonas spp., |
| | Corynebacterium spp., Neisseria gonorrhoeae |
| | herpes simplex; |
| Testicle | Escherichia coli, Klebsiella pneumoniae, Pseudomonas |
| | aeruginosa, Staphylococcus spp., Streptococcus spp., |
| | Salmonella enteriditis |
| | mumps, coxsackievirus, lymphocytic choriomeningitis |
| | virus; |
| Penis | Staphylococcus aureus, Streptococcus pyogenes, |
| | Neisseria gonorrhoeae, Treponema pallidum |
| | herpes simplex, human papillomavirus; |
| Ovary/Adnexae | Neisseria gonorrhoeae, Chlamydia trachomatis, |
| | Gardenerella vaginalis, Prevotella spp., Bacteroides spp., |
| | Peptococcus spp. Streptococcus spp., Escherichia coli; |
| Uterus | Neisseria gonorrhoeae, Chlamydia trachomatis, |
| | Gardenerella vaginalis, Prevotella spp., Bacteroides spp., |
| | Peptococcus spp., Streptococcus spp., Escherichia coli; |
| Cervix | Neisseria gonorrhoeae, Chlamydia trachomatis, |
| | Treponema pallidum |
| | herpes simplex; |
| Vagina | Gardenerella vaginalis, Prevotella spp., Bacteroides spp., |
| | peptococci spp., Escherichia coli, Neisseria gonorrhoeae, |
| | Chlamydia trachomatis, Treponema pallidum, |
| | herpes simplex; |
| Vulva | Staphylococcus aureus, Streptococcus pyogenes, |
| | Treponema pallidum |
| | herpes simplex. |
